(19) United States
(12) Patent Application Publication
(10) Pub. No.: US 2012/0003191 A1
(43) Pub. Date: Jan. 5, 2012

(54) VALPROIC ACID, DERIVATIVES, ANALOGUES, AND COMPOSITIONS INCLUDING SAME AND METHODS FOR THEIR THERAPEUTIC USE

(76) Inventors: Dean J. Burkin, Sparks, NV (US); Praveen B. Gurpur, Bangalore (IN); Stephen J. Kaufman, Urbana, IL (US)

(21) Appl. No.: 13/140,705

(22) PCT Filed: Dec. 18, 2009
(86) PCT No.: PCT/US09/68664

§ 371 Date: Jun. 17, 2011

(51) Int. Cl. A61K 31/19; A61P 21/00; A61K 38/18; A61K 35/12; A61K 38/14; A61K 38/00

(52) U.S. Cl. 424/93.7; 514/557; 514/20.9; 514/1.1; 514/7.6

(57) ABSTRACT

The present disclosure includes methods and compositions for treating any condition or disorder that benefits from activation of the Akt signaling pathway. These methods and compositions involve the use of valproic acid, derivatives, analogs and compositions including the same for treating muscular disorders, such as muscular dystrophy.

### CROSS REFERENCE TO RELATED APPLICATION

This claims the benefit of U.S. Provisional Application No. 61/139,087, filed Dec. 19, 2008, which is incorporated by reference herein in its entirety.

### STATEMENT OF GOVERNMENT SUPPORT

This invention was made with United States Government support under grants from the National Institutes of Health (NIH), Grant No. AG14632, National Institute of Arthritis and Musculoskeletal and Skin Diseases, Grant No. NIAMS R01AR053697, and National Institute of Neurological Disorders and Stroke, Grant No. NINDS R21NS0584291. The United States Government has certain rights in the invention.

### FIELD OF DISCLOSURE

The present disclosure relates to valproic acid and in particular, methods of using valproic acid to ameliorate a condition or disease, such as a muscular disorder, for example, muscular dystrophy.

### BACKGROUND OF DISCLOSURE

Muscular dystrophies are a group of diseases characterized by skeletal muscle degeneration, inflammation, necrosis and fibrosis that lead to progressive muscle weakness. The most common form, Duchenne muscular dystrophy (DMD), is caused by mutations in the gene encoding dystrophin, a member of the dystrophin-glycoprotein complex. This protein complex links laminin in the extracellular matrix to actin in the cytoskeleton. To date, many efforts to cure or ameliorate muscular dystrophy involve enhancing expression of various components of the costameric network. However, these approaches, while showing some promise in vitro or in transgenic animals, typically do not demonstrate effective results in humans.

### SUMMARY OF DISCLOSURE

In various embodiments, the present disclosure provides a method of providing therapeutic benefit by administering to a subject a therapeutically-effective amount of an active agent that includes valproic acid, a valproic acid derivative, a valproic acid analogue, or a mixture thereof. In another embodiment, the present disclosure provides a composition that includes the active agent. In a specific example, the active agent is valproic acid. In another example, the active agent is a salt of valproic acid, such as sodium valproate. The composition, in another example, includes a mixture of valproic acid and a valproic acid salt. In some cases, the effective amount is a dose applied at a dosage regime to provide an Akt activating amount of active agent to the subject.

The active agent, or composition including the active agent, can be administered with other substances, including other therapeutically active substances. In a specific example, the additional therapeutically active substance is a substance that provides therapeutic benefit to a subject suffering from a muscular condition or disease, such as muscular dystrophy (e.g., congenital muscular dystrophy (such as merosin deficient congenital muscular dystrophy (MDC1A), Duchenne muscular dystrophy, or limb-girdle muscular dystrophy). In other examples, the additional therapeutically active substance is a substance that decreases inflammation, apoptosis or prolongs cell survival. In some aspects, the additional therapeutic agent enhances the therapeutic effect of the active agent or active agent composition. In further aspects, the therapeutic agent provides independent therapeutic benefit for the condition being treated. In various examples, the additional therapeutic agent is a component of the extracellular matrix, such as an integrin, laminin, dystrophin, dystroglycan, utrophin, or a growth factor. In further examples, the therapeutic agent reduces or enhances expression of a substance that enhances the formation or maintenance of the extracellular matrix.

Further implementations of the disclosed method include diagnosing the subject as having a condition treatable by activating Akt, such as by administering the active agent or composition thereof. In a particular example, the subject is a human. In further instances, the condition is characterized by the failure of a subject (or the reduced ability of the subject) to express one or more proteins associated with the formation or maintenance of the extracellular matrix, such as impaired or non-production of a laminin, an integrin, dystrophin, utrophin, or dystroglycan. In one example, the subject is diagnosed as suffering from muscular dystrophy, such as a congenital muscular dystrophy (such as merosin deficient congenital muscular dystrophy (MDC1A)), Duchenne muscular dystrophy, or limb-girdle muscular dystrophy. In a specific example, the condition is not characterized by deficient production of the SMN protein. In another example, the active agent or composition is not administered to act as a deacetylase inhibitor. In a further implementation, the method involves diagnosing the subject as suffering from a disease, disorder, or condition characterized by a mutation in the gene encoding α7 integrin. In another implementation, the method involves diagnosing the subject as suffering from a disease, disorder, or condition characterized by a decreased level of α7 integrin expression.

Additional implementations of the disclosed method include selecting a subject in need of treatment. In some examples, the method includes selecting a subject with a muscular disorder including muscular dystrophy. In further examples, the method includes selecting a subject with a disorder associated with inflammation or apoptosis. Identifying a subject in need of treatment can include methods known to those of skill in the art to determine the respective disorder. For example, a subject with muscular dystrophy is identified/selected as one that has increased serum creatine kinase (CK) levels such as at least a 10% increase in serum CK levels as compared to those in a subject without a muscular disorder.

Although the disclosed methods generally have been described with respect to improving the condition of skeletal muscle, the disclosed methods also may be used to enhance the condition of other tissues and organs. For example, the methods of the present disclosure can be used to treat symptoms of muscular dystrophy stemming from effects to cells or tissue other than skeletal muscle, such as impaired or altered brain function, smooth muscles, or cardiac muscles.

There are additional features and advantages of the various embodiments of the present disclosure. They will become evident from the following disclosure.

In this regard, it is to be understood that this is a brief summary of the various embodiments described herein. Any given embodiment of the present disclosure need not provide all features noted above, nor must it solve all problems or address all issues in the prior art noted above.

### BRIEF DESCRIPTION OF THE FIGURES

FIG. 1 is a graph of fluorescence intensity (relative units) versus valproic acid concentration (mM).

FIG. 2 is a an image of a Western analysis of alpha-7 integrin expression, compared with actin control, in extracts of α7^{+/-} myotubes exposed for 48 or 72 hours to 2 mM VPA in duplicate plates.

FIG. 3 is a graph of the intensity of the bands from FIG. 2.

FIG. 4 is phase-contrast micrographs of α7^{+/-} myotubes exposed to valproic acid for 72 hours compared with untreated control cells (upper panel) and myotubes exposed to valproic acid for 72 hours stained with anti-myosin heavy chain antibody (lower panel).

FIG. 5 is a graph of area divided by number of myotubes nuclei for the control and valproic acid treated myotubes of FIG. 4.

FIG. 6 is phase-contrast micrographs of α7^{+/-} myotubes treated with valproic acid for 6 days (starting on day-2 of differentiation) compared with untreated controls (upper panel) and α7^{+/-} myotubes in 8-well chamber slides exposed to either 2 mM valproic acid or no treatment for 6 days using a TUNEL assay (middle panel) to identify FITC-labeled apoptotic nuclei or DAPI staining (lower panel) to identify all nuclei.

FIG. 7 is a graph of TUNEL positive nuclear clusters for the control and valproic acid treated myotubes of FIG. 6.

FIG. 8 is a an image of a Western analysis of day-2 α7^{+/-} myotubes treated with 2 mM valproic acid for 72 or 96 hours using antibodies for the signaling proteins Akt, mTOR, p70S6K, and ERK.

FIG. 9 is a an image of a Western analysis of α7^{+/-} myotubes in duplicate plates treated with 2 mM valproic acid with protein extracted every 24 hours up to 144 hours. The Western blotting identifies phospho and total Akt.

FIG. 10 is a graph of the ratio of phospho Akt to Akt versus valproic acid treatment duration for the myotubes of FIG. 9.

FIG. 11 is phase-contrast micrographs of α7^{+/-} myotube controls and myotubes treated with 2 mM valproic acid with and without 100 nM Wortmannin. Fresh drugs were added every 24 hours.

FIG. 12 is an image of a Western analysis of α7^{+/-} myotubes treated as described in FIG. 11 and immunoblottted for phospho and total Akt.

FIG. 13 is a graph of the ratio of phospho Akt to Akt for the myotubes of FIG. 12.

FIG. 14 is an image of a Western analysis of α7^{+/-} myotubes exposed to various concentrations of valproic acid for 1 hour, followed by extraction and immunoblotting for Akt.

FIG. 15 is a graph of the ratio of phospho Akt to Akt versus valproic acid concentration (mM) for the myotubes of FIG. 14.

FIG. 16 is micrographs of α7^{+/-} myotube controls and myotubes exposed to 30 mM valproic acid for 1 hour with and without Wortmannin after 72 hours.

FIG. 17 is a graph of the prevalence of contractures (%) for saline and valproic acid treated mdx/utrn^{-/-} mice.

FIG. 18 is photomicrographs of cryosections of saline and valproic acid treated mdx/utrn^{-/-} mice quadriceps stained with Masson's trichrome.

FIG. 19 is an image of a Western analysis of SDS extracts of saline and valproic acid treated mdx/utrn^{-/-} mice quadriceps immunoblottted for collagen type VI.

FIG. 20 is a graph of band intensity for the extracts of FIG. 19.

FIG. 21 is photomicrographs of cryosections of saline and valproic acid treated mdx/utrn^{-/-} mice quadriceps injected with Evans blue dye (50 mg/kg) and treated with FITC-wheat germ agglutinin to delineate myofibers.

FIG. 22 is a graph of the percentage of Evans blue positive fibers for saline and valproic acid treated mdx/utrn^{-/-} mice quadriceps cryosections.

FIG. 23 is photomicrographs of cryosections of saline and valproic acid treated mdx/utrn^{-/-} mice quadriceps stained for CD8 positive cytotoxic T-cells using a FITC-labeled anti-CD8a antibody and co-stained with DAPI to delineate nuclei.

FIG. 24 is a graph of the ratio of CD8+ cells to nuclei for saline and valproic acid treated mdx/utrn^{-/-} mice quadriceps stained for CD8 positive cytotoxic T-cells using a FITC-labeled anti-CD8a antibody and co-stained with DAPI to delineate nuclei.

FIG. 25 is an image of a Western analysis of extracts of saline and valproic acid treated mdx/utrn^{-/-} mice quadriceps immunoblottted for activated and total Akt.

FIG. 26 is a graph of the ratio of phospho Akt to Akt for saline and valproic acid treated mdx/utrn^{-/-} mice quadriceps extracts.

### DETAILED DESCRIPTION OF SEVERAL EMBODIMENTS

### I. Abbreviations

Akt—protein kinase B

pAkt—phospho protein kinase B

bFGF—basic fibroblast growth factor

BSA—bovine serum albumin

CD8—cluster of differentiation 8 protein

CK—creatine kinase

DAPI—4′,6-diamidino-2-phenylindole

DMD—Duchenne muscular dystrophy

DMEM—Dulbecco's modified Eagle's medium

EBD—Evan's blue dye

EDTA—ethylenediamine tetra acetic acid

EGTA—ethylene glycol tetraacetic acid

ERK—extracellular signal-regulated kinases

FBS—fetal bovine serum

FCMD—fukuyama congenital muscular dystrophy

FDG—fluorodeoxyglucose

FITC—fluorescein isothiocyanate

FSHD—facioscapulohumeral muscular dystrophy

HBSS—Hank's balanced salt solution

HDAC—histone deacetylase

IGF-1—insulin-like growth factor

mTOR—rapamycin-sensitive kinase mammalian target of rapamycin

pmTOR—phospho rapamycin-sensitive kinase mammalian target of rapamycin

NaPPi—sodium pyrophosphate

P13K—phosphatidyl inositol 3-OH kinase

70S6K—p70S6 kinase

P-p70S6K—phospho p70S6 kinase

PBS—phosphate buffered saline

PDK—phosphoinositide-dependent kinase

P-ERK—phospho extracellular signal-regulated kinases

SD—standard deviation

SDS—sodium dodecyl sulfate

SDS-PAGE—sodium dodecyl sulfate polyacrylamide gel electrophoresis

TBST—tris buffered saline solution with Tween 20

Tris-Cl—a solution made from tris(hydroxymethyl)aminomethane and hydrochloric acid

TUNEL—terminal deoxynucleotidyl transferase mediated dUTP nick end labeling assay

VPA—valproic acid

WGA—wheat-germ agglutinin

### II. Terms

In order to facilitate an understanding of the embodiments presented, the following explanations are provided.

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, or including A and B. All numerical ranges given herein include all values, including end points (unless specifically excluded) and any and all intermediate ranges between the endpoints.

Although methods and materials similar or equivalent to those described herein can be used in the practice or testing of the present disclosure, suitable methods and materials are described herein. The disclosed materials, methods, and examples are illustrative only and not intended to be limiting.

“Administering” refers to providing one or more substances to a subject such that the subject may receive therapeutic benefit from the substance. The active agent, composition, or other therapeutic agent are in general administered topically, nasally, intravenously, orally, intracranially, intramuscularly, parenterally or as implants, but even rectal or vaginal use is possible in principle. The active agent, composition, or other therapeutic agent also may be administered to a subject using a combination of these techniques.

Suitable solid or liquid pharmaceutical preparation forms are, for example, aerosols, (micro)capsules, creams, drops, drops or injectable solution in ampoule form, emulsions, granules, powders, suppositories, suspensions, syrups, tablets, coated tablets, and also preparations with protracted release of active compounds, in whose preparation excipients and additives and/or auxiliaries such as binders, coating agents, disintegrants, flavorings, lubricants, solubilizers, sweeteners, or swelling agents are customarily used as described above. The pharmaceutical compositions are suitable for use in a variety of drug delivery systems. For a brief review of various methods for drug delivery, see Langer, “New Methods of Drug Delivery,” Science 249:1527-1533 (1990), incorporated by reference herein to the extent not inconsistent with the present disclosure.

The active agent, composition, or other therapeutic agents of the present disclosure can be formulated into therapeutically-active pharmaceutical compositions that can be administered to a subject parenterally or orally. Parenteral administration routes include, but are not limited to epidermal, intraarterial, intramuscular (IM and depot IM), intraperitoneal (IP), intravenous (IV), intrasternal injection or infusion techniques, intranasal (inhalation), intrathecal, injection into the stomach, subcutaneous injections (subcutaneous (SQ and depot SQ), transdermal, topical, and ophthalmic.

The active agent, composition, or other therapeutic agent can be mixed or combined with a suitable pharmaceutically acceptable excipients to prepare pharmaceutical compositions. Pharmaceutically acceptable excipients include, but are not limited to, alumina, aluminum stearate, buffers (such as phosphates), glycine, ion exchangers (such as to help control release of charged substances), lecithin, partial glyceride mixtures of saturated vegetable fatty acids, potassium sorbate, serum proteins (such as human serum albumin), sorbic acid, water, salts or electrolytes such as cellulose-based substances, colloidal silica, disodium hydrogen phosphate, magnesium trisilicate, polyacrylates, polyalkylene glycols, such as polyethylene glycol, polyethylene-polyoxypropylene-block polymers, polyvinyl pyrrolidone, potassium hydrogen phosphate, protamine sulfate, group 1 halide salts such as sodium chloride, sodium carboxymethylcellulose, waxes, wool fat, and zinc salts, for example. Liposomal suspensions may also be suitable as pharmaceutically acceptable carriers.

Upon mixing or addition of the active agent, composition, or other therapeutic agent, the resulting mixture may be a solid, solution, suspension, emulsion, or the like. These may be prepared according to methods known to those of ordinary skill in the art. The form of the resulting mixture depends upon a number of factors, including the intended mode of administration and the solubility of the agent in the selected carrier.

Pharmaceutical carriers suitable for administration of the active agent or composition, including any other therapeutic agents, include any such carriers known to be suitable for the particular mode of administration. In addition, the active agent, composition, or additional therapeutic agent can also be mixed with other inactive or active materials that do not impair the desired action, or with materials that supplement the desired action, or have another action.

Methods for solubilizing may be used where the agents exhibit insufficient solubility in a carrier. Such methods are known and include, but are not limited to, dissolution in aqueous sodium bicarbonate, using cosolvents such as dimethylsulfoxide (DMSO), and using surfactants such as TWEEN® (ICI Americas, Inc., Wilmington, Del.).

The active agent, composition, or other therapeutic agent can be prepared with carriers that protect them against rapid elimination from the body, such as coatings or time-release formulations. Such carriers include controlled release formulations, such as, but not limited to, microencapsulated delivery systems. The active agent, composition, or other therapeutic agent is included in the pharmaceutically acceptable carrier in an amount sufficient to exert a therapeutically useful effect, typically in an amount to avoid undesired side effects, on the treated subject. The therapeutically effective concentration may be determined empirically by testing the compounds in known in vitro and in vivo model systems for the treated condition. For example, mouse models of muscular dystrophy may be used to determine effective amounts or concentrations that can then be translated to other subjects, such as humans, as known in the art.

Injectable solutions or suspensions can be formulated, using suitable non-toxic, parenterally-acceptable diluents or solvents, such as 1,3-butanediol, isotonic sodium chloride solution, mannitol, Ringer's solution, saline solution, or water; or suitable dispersing or wetting and suspending agents, such as sterile, bland, fixed oils, including synthetic mono- or diglycerides, and fatty acids, including oleic acid; a naturally occurring vegetable oil such as coconut oil, cottonseed oil, peanut oil, sesame oil, and the like; glycerine; polyethylene glycol; propylene glycol; or other synthetic solvent; antimicrobial agents such as benzyl alcohol and methyl parabens; antioxidants such as ascorbic acid and sodium bisulfite; buffers such as acetates, citrates, and phosphates; chelating agents such as ethylenediaminetetraacetic acid (EDTA); agents for the adjustment of tonicity such as sodium chloride and dextrose; and combinations thereof. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate-buffered saline (PBS), and solutions containing thickening and solubilizing agents such as glucose, polyethylene glycol, polypropyleneglycol, and mixtures thereof. Liposomal suspensions, including tissue-targeted liposomes, may also be suitable as pharmaceutically acceptable carriers.

For topical application, the active agent, composition, or other therapeutic agent may be made up into a cream, lotion, ointment, solution, or suspension in a suitable aqueous or non-aqueous carrier. Topical application can also be accomplished by transdermal patches or bandages which include the therapeutic substance. Additives can also be included, e.g., buffers such as sodium metabisulphite or disodium edetate; preservatives such as bactericidal and fungicidal agents, including phenyl mercuric acetate or nitrate, benzalkonium chloride, or chlorhexidine; and thickening agents, such as hypromellose.

If the active agent, composition, or other therapeutic agent is administered orally as a suspension, the pharmaceutical compositions can be prepared according to techniques well known in the art of pharmaceutical formulation and may contain a suspending agent, such as alginic acid or sodium alginate, bulking agent, such as microcrystalline cellulose, a viscosity enhancer, such as methylcellulose, and sweeteners/flavoring agents. Oral liquid preparations can contain conventional additives such as suspending agents, e.g., gelatin, glucose syrup, hydrogenated edible fats, methyl cellulose, sorbitol, and syrup; emulsifying agents, e.g., acacia, lecithin, or sorbitan monooleate; non-aqueous carriers (including edible oils), e.g., almond oil, fractionated coconut oil, oily esters such as glycerine, propylene glycol, or ethyl alcohol; preservatives such as methyl or propyl p-hydroxybenzoate or sorbic acid; and, if desired, conventional flavoring or coloring agents. When formulated as immediate release tablets, these compositions can contain dicalcium phosphate, lactose, magnesium stearate, microcrystalline cellulose, and starch and/or other binders, diluents, disintegrants, excipients, extenders, and lubricants.

If oral administration is desired, the active agent, composition, or other therapeutic substance can be provided in a composition that protects it from the acidic environment of the stomach. For example, the active agent, composition, or other therapeutic agent can be formulated with an enteric coating that maintains its integrity in the stomach and releases the active compound in the intestine. The active agent, composition, or other therapeutic agent can also be formulated in combination with an antacid or other such ingredient.

Oral compositions generally include an inert diluent or an edible carrier and can be compressed into tablets or enclosed in gelatin capsules. For the purpose of oral therapeutic administration, the active agent, composition, or other therapeutic substance can be incorporated with excipients and used in the form of capsules, tablets, or troches. Pharmaceutically compatible adjuvant materials or binding agents can be included as part of the composition.

The capsules, pills, tablets, troches, and the like can contain any of the following ingredients or compounds of a similar nature: a binder such as, but not limited to, acacia, corn starch, gelatin, gum tragacanth, polyvinylpyrrolidone, or sorbitol; a filler such as calcium phosphate, glycine, lactose, microcrystalline cellulose, or starch; a disintegrating agent such as, but not limited to, alginic acid and corn starch; a lubricant such as, but not limited to, magnesium stearate, polyethylene glycol, silica, or talc; a gildant, such as, but not limited to, colloidal silicon dioxide; a sweetening agent such as sucrose or saccharin; disintegrants such as potato starch; dispersing or wetting agents such as sodium lauryl sulfate; and a flavoring agent such as peppermint, methyl salicylate, or fruit flavoring.

When the dosage unit form is a capsule, it can contain, in addition to material of the above type, a liquid carrier, such as a fatty oil. In addition, dosage unit forms can contain various other materials that modify the physical form of the dosage unit, for example, coatings of sugar and other enteric agents. The active agent, composition, or other therapeutic agent can also be administered as a component of an elixir, suspension, syrup, wafer, tea, chewing gum, or the like. A syrup may contain, in addition to the active compounds, sucrose or glycerin as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, they can be of the sustained release type so that the compounds need to be administered less frequently.

As used herein, the term “AKT” refers to the AKT protein family, which includes protein kinase B (PKB), and plays a role in mammalian cellular signaling. In humans, there are three genes in the “Akt family”: Akt1, Akt2, and Akt3. These genes code for enzymes that are members of the serine/threonine-specific protein kinase family. Akt1 is involved in cellular survival pathways, by inhibiting apoptotic processes. Akt1 is also able to induce protein synthesis pathways, and is therefore a signaling protein in the cellular pathways that leads to skeletal muscle hypertrophy, and general tissue growth. Akt2 is signaling molecule in the insulin signaling pathway. It is required to induce glucose transport. Akt3 is predominantly expressed in brain.

Akt possesses a protein domain known as a pleckstrin homology (PH) domain. This domain binds to phosphoinositides with high affinity. In the case of the PH domain of Akt, it binds either phosphatidylinositol (3,4,5)-trisphosphate (PtdIns(3,4,5)P₃ aka PIP₃) or phosphatidylinositol (3,4)-bisphosphate (PtdIns(3,4)P₂ aka PI(3,4)P₂). This is useful for control of cellular signaling because the diphosphorylated phosphoinositide PtdIns(4,5)P₂ is only phosphorylated by the family of enzymes, PI 3-kinases (phosphoinositide 3-kinase or PI3K), and only upon receipt of chemical messengers which tell the cell to begin the growth process. For example, PI 3-kinases may be activated by a G protein coupled receptor or receptor tyrosine kinase such as the insulin receptor. Once activated, PI 3-kinases phosphorylates PtdIns(4,5)P₂ to form PtdIns(3,4,5)P₃.

Once correctly positioned in the membrane via binding of PIP3, Akt can be phosphorylated by its activating kinases, phosphoinositide dependent kinase 1 (PDPK1 at threonine 308) and mTORC2 (at serine 473). Phosphorylation by mTORC2 stimulates the subsequent phosphorylation of Akt by PDK1. Activated Akt can activate or deactivate its myriad substrates via its kinase activity. Besides being a downstream effector of PI 3-kinases, Akt may also be activated in a PI 3-kinase-independent manner. Studies have suggested that cAMP-elevating agents can activate Akt through protein kinase A (PKA).

PIP3 can also be de-phosphorylated at the “5” position by the SHIP family of inositol phosphatases, SHIP1 and SHIP2. These poly-phosphate inositil phosphatases dephosphorylate PtdIns(3,4,5)P3 to form PtdIns(3,4)P2. The phosphatases in the PHLPP family, PHLPP1 and PHLPP2 have been shown to directly de-phosphorylate, and therefore inactivate, distinct Akt isoforms. PHLPP2 dephosphorylates Akt1 and Akt3, whereas PHLPP1 is specific for Akt 2 and Akt3. Akt regulates cellular survival and metabolism by binding and regulating many downstream effectors, e.g., nuclear Factor-κB, Bcl-2 family proteins and murine double minute 2 (MDM2).

In particular examples, VPA, a VPA analogue, or a VPA derivative activates Akt. For example, VPA activation results in a decrease in muscular inflammation, apoptosis, muscle loss or serum CK levels. Akt activation is a treatment for muscular disorders, including muscular dystrophy.

Nucleic acid and protein sequences for Akt are publicly available. For example, GenBank Accession Nos.: Akt1: NG_{—}012188; Akt2: BC063421; Akt3: AF135794 disclose Akt1 nucleic acid sequences, and GenBank Accession Nos.: Akt1: AAL55732; Akt2: NP_{—}001617; Akt3: AAH20479 disclose Akt1 protein sequences, all of which are incorporated by reference as provided by GenBank on Dec. 18, 2009

As used herein, the term “ameliorating,” with reference to a condition, refers to any observable beneficial effect of the treatment. The beneficial effect can be evidenced, for example, by a delayed onset of clinical symptoms of the condition in a susceptible subject, a reduction in severity of some or all clinical symptoms of the condition, a slower progression of the condition, a reduction in the number of relapses of the condition, an improvement in the overall health or well-being of the subject, by other parameters well known in the art that are specific to the particular condition, and combinations of such factors. For example, ameliorating, in some embodiments of the disclosed method, refers to delaying progression of muscular dystrophy or eliminating or reducing the severity of one or more muscular dystrophy symptoms.

As used herein, an “analog” or “analogue” refers to a compound which is sufficiently homologous or structurally and chemically similar to a compound such that it has a similar functional activity for a desired purpose as the original compound. Analogues include polypeptides having one or more amino acid substitutions compared with a particular substance. Analogues of valproic acid include valnoctamide and those disclosed in U.S. Patent Publication 2006/0223888, incorporated by reference herein to the extent not inconsistent with the present disclosure.

“At least substantially homologous,” as used in the present disclosure, refers to a degree of homology sufficient to produce at least a portion of the activity of a reference material, such as Akt activation. In some examples, materials are at least substantially homologous when they are at least about 95%, at least about 98%, or at least about 99% homologous to a reference material.

“Apoptosis” refers to a process of cellular suicide. Apoptosis is one of the main types of programmed cell death, and involves an orchestrated series of biochemical events leading to a characteristic cell morphology and death. The apoptotic process is executed in such a way as to safely dispose of cellular debris.

In contrast to necrosis, which is a form of traumatic cell death that results from acute cellular injury, apoptosis is carried out in an orderly process that generally confers advantages during an organism's life cycle. For example, the differentiation of fingers and toes in a developing human embryo requires cells between the fingers to initiate apoptosis so that the digits can separate. Between 50 billion and 70 billion cells die each day due to apoptosis in the average human adult. For an average child between the ages of 8 to 14, approximately 20 billion to 30 billion cells die a day.

Defective apoptotic processes have been implicated in an extensive variety of diseases. Excessive apoptosis causes hypotrophy, such as in ischemic damage, whereas an insufficient amount results in uncontrolled cell proliferation, such as cancer. In one particular example, valproic acid or a derivative thereof is used to inhibit or treat one or more signs or symptoms associated with apoptosis.

“Biological source” refers to an organism, such as an animal, such as a mammal, or portion thereof, from which biological materials may be obtained. Examples of such materials include tissue samples, such as placental material or sarcoma; cells, such as satellite cells; extracellular material, including laminins or other components thereof; or other organic or inorganic material found in the organism.

A “derivative,” as used herein, refers to a form of a substance, such as valproic acid, which has at least one functional group altered, added, or removed, compared with the parent compound. Derivatives include, for example, esterified acids and salts. Thus, sodium valproate is a derivative of valproic acid.

In some cases derivatives may be a prodrug of the active agent. For example esters and amine derivatives of valproic acid may be bioconverted to valproic acid. Examples of such derivatives include valpromide, butyl valproate, hexyl valproate, isoamyl valproate, isobutyl valproate, propyl valproate sodium valproate, 2-propylpentanol-di-n-propylacetate, glycerol tri-dipropylacetate, di sodium valproate, and 1′-ethoxycarbonyloxyethyl ester of valproic acid. Prodrugs may be beneficial for a number of reasons, including enhanced solubility, lower toxicity, or for use in extended release compositions.

“Effective amount” refers to an amount effective for lessening, ameliorating, eliminating, preventing, or inhibiting at least one symptom of a disease, disorder, or condition treated and may be empirically determined. In various embodiments of the present disclosure, a “therapeutically-effective amount” is a “muscle regeneration promoting-amount,” an amount sufficient to achieve a statistically significant promotion of tissue or cell regeneration, such as muscle cell regeneration, compared to a control. For example, a therapeutically-effective amount is an amount sufficient to increase tissue or cell regeneration by at least a 10%, 20%, 30%, 50%, 70%, 80%, 90%, or 95% as compared to a control (e.g., as compared to levels of tissue regeneration prior to treatment or in a subject afflicted with the disorder, but not receiving the treatment). In another example, a “therapeutically-effective amount” is an “Akt-activating” amount, an amount sufficient to achieve a level of Akt activation sufficient to produce a statistically significant effect, such as an ameliorative effect, with respect to a disease, disorder, or condition responsive to treatment by Akt activation.

In some examples, a therapeutically effective amount is an amount of a composition sufficient to achieve a desired biological effect, for example an amount that is effective to reduce one or more signs or symptoms associated with any muscular disorder and/or condition, including muscular dystrophy. For example, a therapeutically effective amount is an amount of a composition that prevents, slows or inhibits the loss of muscle mass such as reduces the loss of muscle mass by at least 10%, 20%, 30%, 50%, 70%, 80%, 90%, or 95% as compared to a control (e.g., as compared to the rate of muscle mass loss prior to treatment or in a subject afflicted with the disorder, but not receiving the treatment).

In particular, indicators of muscular health, such as muscle cell regeneration, maintenance, or repair, can be assessed through various means, including monitoring markers of muscle regeneration, such as transcription factors such as Pax7, Pax3, MyoD, MRF4, and myogenin. For example, increased expression of such markers can indicate that muscle regeneration is occurring or has recently occurred. Markers of muscle regeneration, such as expression of embryonic myosin heavy chain (eMyHC), can also be used to gauge the extent of muscle regeneration, maintenance, or repair. For example, the presence of eMyHC can indicate that muscle regeneration has recently occurred in a subject.

Muscle cell regeneration, maintenance, or repair can also be monitored by determining the girth, or mean cross sectional area, of muscle cells or density of muscle fibers. Additional indicators of muscle condition include muscle weight and muscle protein content. Mitotic index (such as by measuring BrdU incorporation) and myogenesis can also be used to evaluate the extent of muscle regeneration.

Activation of the Akt pathway can be monitored by looking for an increase in Akt, mTOR, or p70S6K. The effect of the active agent may also be monitoring for a decrease in ERK, collagen, EBD uptake, or CD8+ cells. Physical signs of Akt activation include myotube hypertrophy, inhibition of apoptosis, and increased myofibers integrity.

In particular examples, the improvement in muscle condition, such as regeneration, compared with a control is at least about 10%, such as at least about 30%, or at least about 50% or more.

In some implementations, the effective amount of active agent or composition thereof is administered as a single dose per time period, such as every day or week, or it can be divided into at least two unit dosages for administration over a period, such as twice daily or twice weekly. Treatment may be continued as long as necessary to achieve the desired results. For instance, treatment may continue for about 3 or 4 days up to about 1 or 2 weeks or longer, including ongoing treatment. The compound can also be administered in several doses intermittently, such as every few days (for example, at least about every two, three, four, five, or ten days) or every few weeks (for example at least about every two, three, four, five, or ten weeks).

Particular dosage regimens can be tailored to a particular subject, condition to be treated, or desired result. For example, when the methods of the present disclosure are used to treat muscular dystrophy, an initial treatment regimen can be applied to arrest the condition. Such initial treatment regimen may include administering a higher dosage of the active agent or composition, or administering such material more frequently, such as hourly. After a desired therapeutic result has been obtained, such as a desired level of symptom reduction, a second treatment regimen may be applied, such as administering a lower dosage of active agent or composition or administering such material less frequently, such as twice daily, daily, or weekly. In such cases, the second regimen may serve as a “booster” to restore or maintain a desired level of activity, such as Akt activation.

Amounts effective for various therapeutic treatments of the present disclosure may, of course, depend on the severity of the condition and the weight and general state of the subject, as well as the absorption, inactivation, and excretion rates of the therapeutically-active compound or component, the dosage schedule, and amount administered, as well as other factors known to those of ordinary skill in the art. It also should be apparent to one of ordinary skill in the art that the exact dosage and frequency of administration will depend on the particular active agent or composition, or any additional therapeutic substance, being administered, the particular condition being treated, the severity of the condition being treated, the age, weight, general physical condition of the particular subject, and other medication the subject may be taking. Typically, dosages used in vitro may provide useful guidance in the amounts useful for in vivo administration of the pharmaceutical composition, and animal models may be used to determine effective dosages for treatment of particular disorders. For example, mouse or guinea pig models of the disease, disorder, or condition to be treated, such as muscular dystrophy, may be used to determine effective dosages that can then be translated to dosage amount for other subjects, such as humans, as known in the art. Various considerations in dosage determination are described, e.g., in Gilman et al., eds., Goodman And Gilman's: The Pharmacological Bases of Therapeutics, 8th ed., Pergamon Press (1990); and Remington's Pharmaceutical Sciences, 17th ed., Mack Publishing Co., Easton, Pa. (1990), each of which is herein incorporated by reference to the extent not inconsistent with the present disclosure.

In specific examples, the active agent or composition is administered to a subject in an amount sufficient to provide a dose active agent of between about 100 μg/kg and about 5000 mg/kg, between about 1 mg/kg and about 2000 mg/kg, between about 100 mg/kg and about 1500 mg/kg, between about 200 mg/kg and about 1000 mg/kg, between about 200 mg/kg and about 750 mg/kg, or between about 250 mg/kg and about 500 mg/kg. In further examples, the active agent or composition is administered to a subject in an amount sufficient to provide a concentration of active agent in the administrated material of between about 100 μM and about 500 mM, such as between about 1 mM and about 100 mM, or between about 5 mM and about 50 mM. In particular examples, one of the preceding amounts is provided twice per day. In another example, this amount is provided more than twice a day, such as three or four times per day. In another example, the active agent or composition is administered in a controlled released manner that provides an effective daily dose corresponding to one of these dosages. Controlled release preparations of valproic acid are disclosed, for example, in U.S. Pat. No. 4,913,906, which is hereby incorporated by reference in its entirety.

“Extracellular matrix” refers to the extracellular structure of a tissue or a layer thereof, including the arrangement, composition, and forms of one or more matrix components, such as proteins, including structural proteins such as collagen and elastin, proteins such as fibronectin and laminins, and proteoglycans. The matrix may include fibrillic collagen, having a network of fibers. In some examples, the extracellular matrix is connected to cells through the costameric protein network.

“Facioscapulohumeral muscular dystrophy” (FSHD) refers to a muscular dystrophy that initially affects muscles of the face, shoulders, and upper arms with progressive weakness. Symptoms usually develop in the teenage years. Some affected individuals become severely disabled. The pattern of inheritance is autosomal dominant. Most cases are associated with a deletion near the end of chromosome 4. In an example, one or more signs or symptoms associated with FSHD is prevented, reduced or inhibited by administration of valproic acid, a valoproic analogue or derivative thereof (such as one or more signs or symptoms are reduced by at least 10%, at least 30%, at least 50%, or at least 70% as compared to a control).

“Fukuyama congenital muscular dystrophy” (FCMD) refers to a muscular dystrophy that is characterized by hypotonia, symmetrical generalized muscle weakness, and CNS migration disturbances that result in changes consistent with cobblestone (previously type II) lissencephaly with cerebral and cerebellar cortical dysplasia. Mild, typical, and severe phenotypes are recognized. Onset typically occurs in early infancy, with a poor suck, weak cry, and floppiness. Affected individuals have contractures of the hips, knees, and interphalangeal joints. Later features include myopathic facial appearance; pseudohypertrophy of the calves and forearms; motor, mental, and speech retardation; convulsions; ophthalmologic abnormalities including visual impairment and retinal dysplasia; and progressive cardiac involvement in individuals over ten years of age. Swallowing disturbance occurs in individuals with severe FCMD and in individuals over ten years of age, leading to recurrent aspiration pneumonia and death. In one example, one or more symptoms associated with FCMD is prevented, reduced or inhibited by administration of valproic acid, a valoproic analogue or derivative thereof. A subject can be diagnosed with FCMD by identifying any of the aforementioned characteristics of the condition including by neuroimaging, electromyography, measurement of serum creatine kinase concentration, muscle biopsy, and molecular genetic testing. FCMD is the only gene known to be associated with FCMD. Molecular genetic testing of the FCMD gene is available on a clinical basis For example, FCMD is identified by neuroimaging by an MRI revealing the findings of cobblestone lissencephaly including the following five major abnormalities: (1) irregular or pebbled brain surface, broad gyri with a thick cortex (pachygyria) in the frontal, parietal, and temporal regions, and sometimes areas of small and irregular gyri that resemble polymicrogyria; (2) dilated lateral ventricles; (3) white matter abnormality with hyperintensity on T2-weighted images and hypointensity on T1-weighted images indicative of dysmyelination; (4) mild brainstem hypoplasia; and (5) cerebellar polymicrogyria and cerebellar cysts. FCMD can also be diagnosed by measuring serum creatine kinase (CK) concentration in which CK levels 10-60 times higher than normal are indicative of muscular dystrophy and possibly, FCMD.

“Functional group” refers to a radical, other than a hydrocarbon radical, that adds a physical or chemical property to a substance.

“Impaired” a term used to describe a reduced or diminished production or response, such as a reduction in quality, strength or utility. In one example, impaired production of a component of the muscle membrane-cytoskeleton-extracellular matrix complexes is an at least 10%, 20%, 30%, 50%, 70%, 80%, 90%, 95% or more reduction in such components.

“Improving muscular health” refers to an improvement in muscular health compared with a preexisting state or compared with a state which would occur in the absence of treatment. For example, improving muscular health may include enhancing muscle regeneration, maintenance, or repair Improving muscular health may also include prospectively treating a subject to prevent or reduce muscular damage or injury. In some example, improving muscular health includes enhancing muscle regeneration, maintenance or repair by at least 10%, such as at least 20%, at least 30%, at least 50%, or at least 70% as compared to muscular health prior to treatment.

“Inflammation” is a complex biological response of vascular tissues to harmful stimuli, such as pathogens, damaged cells, or irritants. Inflammation is a protective attempt by the organism to remove the injurious stimuli. This generalized response by the body includes the release of many components of the immune system (for instance, IL-1 and TNF), attraction of cells to the site of the damage, swelling of tissue due to the release of fluid and other processes.

“Inhibiting” refers to a full or partial reduction in the development or progression of the condition, for example, in a subject who is at risk for a condition or who has a particular condition, such as a muscular disorder, for example, muscular dystrophy. Particular methods of the present disclosure provide methods for inhibiting muscular dystrophy. For example, methods are disclosed herein for inhibiting one or more symptoms associated with a muscular disorder, including muscular dystrophy. In some examples, inhibition includes a partial reduction in one or more symptoms such as at least a 10%, 20%, 30%, 50%, 70%, 80%, 90%, 95% or complete elimination in the one or more symptoms as compared to a control (e.g., as compared to the rate of the one or more symptoms prior to treatment or in a subject afflicted with the disorder, but not receiving the treatment).

“Laminin” refers to any of the family of glycoproteins that are typically involved in the formation and maintenance of extracellular matrices. Laminin is a heterotrimers formed from an α chain, a β chain, and γ chain. The various chains of a particular laminin can affect the properties of the molecule. In some aspects of the present disclosure, fragments, derivatives, or analogs of various laminins can be used, such as laminins having at least a portion at least substantially homologous to the laminin α1 chain.

“Maintenance” of cells or tissue, such as muscle cells or tissue (or organs) which includes muscle cells, refers to maintaining the cells or tissue in at least substantially the same physiological condition, such as maintaining such condition even in the presence of stimulus which would normally cause damage, injury, or disease.

“Merosin deficient congenital muscular dystrophy” (MDC1A) is the most common type of congenital muscular dystrophies (CMDs), accounting for 30-40% of the CMDs. MDC1A is caused by a mutation in the lama2 gene which encodes the laminin α2 chain Laminins are heterotrimeric proteins composed of a heavy α chain and two structurally similar light chains (β and γ) and are a major component of the extracellular matrix (ECM). Laminin 111 (α1, β1, γ1) is the predominant isoform found in developing skeletal muscle with laminin 211 (α2, β1, γ1) being the predominant isoform in differentiated skeletal muscle. In an example, one or more signs or symptoms associated with MDC1A is prevented, reduced or inhibited by administration of valproic acid, a valoproic analogue or derivative thereof (such as one or more signs or symptoms are reduced by at least 10%, at least 30%, at least 50%, or at least 70% as compared to a control).

“Muscle” refers to any myoblast, myocyte, myofiber, myotube or other structure composed of muscle cells. Muscles or myocytes can be skeletal, smooth, or cardiac. Muscle may also refer to, in particular implementations of the present disclosure, cells or other materials capable of forming myocytes, such as stem cells and satellite cells.

“Regeneration” refers to the repair of cells or tissue, such as muscle cells or tissue (or organs) which includes muscle cells, following injury or damage to at least partially restore the muscle or tissue to a condition similar to which the cells or tissue existed before the injury or damage occurred. Regeneration also refers to facilitating repair of cells or tissue in a subject having a disease affecting such cells or tissue to eliminate or ameliorate the effects of the disease. In more specific examples, regeneration places the cells or tissue in the same condition or an improved physiological condition as before the injury or damage occurred or the condition which would exist in the absence of disease.

“Repair” of cells or tissue, such as muscle cells or tissue (or organs) which includes muscle cells, refers to the physiological process of healing damage to the cells or tissue following damage or other trauma.

“Subject” refers to an organism, such as an animal, to which treatments are administered. Subjects include mammals, such as humans, pigs, rats, cows, mice, dogs, cats, and primates.

“Survival (of a Cell)” refers to the length of time a given cell is alive. An increase in survival following treatment indicates that the cell lives for a longer length of time as compared to a control, such as the cell in the absence of treatment. In one example, valproic acid or an analogue or derivative thereof increases cell survival by at least 10%, such as by at least 20%, at least 30%, at least 40%, at least 50%, at least 60%, at least 70%, at least 80%, at least 90%, or more as compared to cell survival in the absence of treatment or prior to treatment. In one particular example, the cell is a muscle cell.

“Tissue” refers to an aggregate of cells, usually of a particular kind, together with their intercellular substance that form one of the structural materials of an animal and that in animals include connective tissue, epithelium, muscle tissue, and nerve tissue.

“Treatment” refers to a therapeutic intervention that ameliorates a sign or symptom of a condition after it has begun to develop. For example, in some embodiments, the method of the present disclosure involves treating muscular dystrophy, such as to reduce one or more symptoms of muscular dystrophy.

“Valproic acid” (VPA) is a branched chain fatty acid that is FDA approved for treating epilepsy and bipolar disorders. VPA is known to have histone deacetylase (HDAC) inhibitor activity. Valproic acid is used herein to prevent, reduce or inhibit one or more symptoms associated with a muscular disorder, including one or more symptoms associated with muscular dystrophy. In one example, valproic acid activates the Akt pathway thereby treating conditions responsive to Akt activation. In some examples, valproic acid is used herein to inhibit inflammation or one or more conditions associated with inflammation. In further examples, valproic acid is used herein to treat apoptosis or one or more signs and symptoms associated with apoptosis. In even further examples, valproic acid is used herein to increase cell survival.

The above term descriptions are provided solely to aid the reader, and should not be construed to have a scope less than that understood by a person of ordinary skill in the art or as limiting the scope of the appended claims.

### III. Methods of Treatment

It is shown herein that valproic acid activates the Akt pathway. Based on these observations, methods of treatment for a condition, disease or disorder which benefits from activation of the Akt pathway are disclosed. Generally, the present disclosure provides embodiments of a method for activating the Akt pathway in a subject by administering to the subject a therapeutically effective amount of an active agent or composition of the active agent. The active agent is selected from valproic acid, a valproic acid derivative, or a valproic acid analogue. Such conditions include muscular dystrophies, such as Duchenne muscular dystrophy, facioscapulohumeral muscular dystrophy (FSHD), Fukyama congenital muscular dystrophy (FCMD), merosin deficient congenital muscular dystrophy (MDC1), limb-girdle muscular dystrophies (LGMD), alpha7 integrin congenital, secondary muscular dystrophies, any muscular dystrophy resulting from defects or deficiencies in any component of the dystrophin glycoprotein complex, and any muscle disease arising from defects or deficiencies in any component of the extracellular matrix. Benefits from such treatment can include reduced muscle inflammation, reduced collagen production, reduced myotube hypertrophy, reduced apoptosis, enhanced myofibers stability and enhanced/increased cell survival. Thus, the treatment can increase the cellular health, including muscular health of the treated subject. Reduction in muscle inflammation, collagen production, myotube hypertrophy, apoptosis or enhanced myofibers stability and cell survival can be measured by techniques known to those of ordinary skill in the art including those described herein.

In one embodiment, administration of valproic acid, a valproic acid derivative or a valproic acid analogue, decreases the serum CK levels in a subject. For example, the disclosed methods can reduce serum CK levels, for example, by at least 10%, at least 20%, at least 30%, at least 40%, at least 50%, such as about 5% to about 90%, including about 10% to about 70% percent, about 20% to about 50% (e.g., 10%, 20%, 30%, 40%, 50%, 60%, 70%, 80% or 90%) as compared to serum CK levels prior to treatment or a standard reference value known to be indicative of a disorder or condition that can benefit from activation of the Akt pathway, such as muscular dystrophy.

In some embodiments, following the administration of one or more therapies, subjects having a condition, disease or disorder which benefits from activation of the Akt pathway (for example, muscular dystrophy) are monitored to determine the response of their muscular tissue to the therapy. For example, subjects are monitored to determine if the therapy resulted in a reduction in muscle inflammation, collagen production, myotube hypertrophy, apoptosis or and/or enhanced myofibers stability and cell survival. In particular examples, subjects are analyzed one or more times, starting 7 days following treatment. Subjects can be monitored using any method known in the art including MRI or indirectly, for example, by measuring changes in the levels of serum CK.

In particular examples, if subjects are stable or have a minor, mixed or partial response to treatment, they can be re-treated after re-evaluation with the same schedule and preparation of compositions that they previously received for the desired amount of time, such as for at least three months, at least six months, at least twelve months, or at least twenty-four months of total treatment. For example, a partial response is one in which a reduction in serum CK levels is observed, but the subject still experiences muscle loss.

Diagnosing and/or Selecting a Subject for Treatment

In additional aspects, the method involves diagnosing the subject as suffering from a condition, disease, or disorder responsive to Akt activation. For example, the method can include diagnosing the subject as suffering from muscular dystrophy, such as Duchenne muscular dystrophy. In some implementations, the active agent or composition is not administered for deacetylase activity or to increase SMN production.

In a further implementation, the method involves diagnosing the subject as suffering from a disease, disorder, or condition characterized by a mutation in the gene encoding α7 integrin. In another implementation, the method involves diagnosing the subject as suffering from a disease, disorder, or condition characterized by a decreased level of α7 integrin expression. Although the present disclosure is not dependent on this mechanism, activation of the Akt pathway may compensate for reduced activation of the Akt pathway resulting from inadequate activation of integrin-linked kinase by α7 integrin.

In some embodiments, the method involves diagnosing a subject with a disorder associated with inflammation or apoptosis. For example, a subject with increased expression of CD4 or CD8, such as at least a 2-fold increase in expression of CD4 or CD8, indicates that such a subject may benefit from the disclosed treatment. For example, a subject with an increased number of CD4 and/or CD8 positive cells indicates that such subject may benefit from the current treatment.

In certain embodiments, the method involves selecting a subject with a muscular disorder. For example, the method includes selecting a subject with muscular dystrophy, such as DMD, FSHD, FCMD or MDC1A.

### Combination Therapy

The active agent may be administered with, or the composition may include, an additional therapeutic agent, which may be used to enhance the properties of the active agent, provide independent therapeutic effect, or improve the pharmacological or biological properties of the active agent, such as reducing its toxicity. In various examples of the embodiments of the present disclosure, the active agent or composition is administered with one or more other components, such as components of the extracellular matrix. In some examples, the additional active agent is a laminin, creatine, or a mixture thereof. The additional substance can also include aggrecan, angiostatin, cadherins, collagens (including collagen I, collagen III, or collagen IV), decorin, elastin, enactin, endostatin, fibrin, fibronectin, osteopontin, tenascin, thrombospondin, vitronectin, and combinations thereof. Biglycans, glycosaminoglycans (such as heparin), glycoproteins (such as dystroglycan), proteoglycans (such as heparan sulfate), and combinations thereof can also be administered.

Growth stimulants may be added in conjunction with the active agent or composition. Examples of growth stimulants include cytokines, polypeptides, and growth factors such as brain-derived neurotrophic factor (BDNF), CNF (ciliary neurotrophic factor), EGF (epidermal growth factor), FGF (fibroblast growth factor), glial growth factor (GGF), glial maturation factor (GMF) glial-derived neurotrophic factor (GDNF), hepatocyte growth factor (HGF), insulin, insulin-like growth factors, kerotinocyte growth factor (KGF), nerve growth factor (NGF), neurotropin-3 and -4, PDGF (platelet-derived growth factor), vascular endothelial growth factor (VEGF), and combinations thereof.

Additional therapeutic agents can be added to enhance the therapeutic effect of the active agent or composition. For example, a source of muscle cells can be added to aid in muscle regeneration and repair. In some aspects of the present disclosure, satellite cells are administered to a subject in combination with the active agent or composition. U.S. Patent Publication 2006/0014287, incorporated by reference herein to the extent not inconsistent with the present disclosure, provides methods of enriching a collection of cells in myogenic cells and administering those cells to a subject.

In further aspects, stem cells, such as adipose-derived stem cells, are administered to the subject. Suitable methods of preparing and administering adipose-derived stem cells are disclosed in U.S. Patent Publication 2007/0025972, incorporated by reference herein to the extent not inconsistent with the present disclosure. Additional cellular materials, such as fibroblasts, can also be administered, in some examples.

The active agents or compositions thereof may be delivered as discrete molecules or may be complexed with, or conjugated to, another substance. For example, the active agent or composition may be combined with a carrier, such as to aid in delivery of the active agent or composition to a site of interest or to increase physiological uptake or incorporation of the active agent or composition.

### Administration of Therapeutically Effective Amount of VPA

A therapeutically effective amount of VPA, a VPA analogue, or a VPA derivative, or a composition including such, can be administered locally or systemically using methods known in the art, to subjects having condition or disorder that benefits from activation of the Akt pathway.

In one embodiment, the VPA, a VPA analogue, or VPA derivative or composition including such is administered systemically, for example intravenously, intramuscularly, subcutaneously, or orally, to a subject having a muscular disorder or a sign or symptom associated with a muscular disorder. A therapeutically effective amount of VPA, a VPA analogue, or a VPA derivative refers to an amount sufficient to achieve a desired biological effect, for example an amount that is effective to decrease serum CK levels, or muscle loss, or decrease symptoms of a muscular disorder. In one embodiment, it is an amount sufficient to decrease the signs or symptoms of muscular dystrophy in a subject. In particular examples, it is an amount effective to reduce a sign or symptom of muscular dystrophy, such as a decrease in serum CK levels, by at least 10%, 20%, 30%, 40%, or 50%. In another embodiment, it is an amount sufficient to prevent further muscle loss. Effective doses can be extrapolated from dose-response curves derived from in vitro or animal model test systems.

An effective amount of a VPA, a VPA analogue, or VPA derivative can be administered in a single dose, or in several doses, for example daily, during a course of treatment. However, the effective amount of VPA, a VPA analogue, or VPA derivative will be dependent on the subject being treated, the severity and type of the condition being treated, and the manner of administration. In one embodiment, a therapeutically effective amount of VPA, a VPA analogue, or VPA derivative can vary from about 100 μg/kg and about 5000 mg/kg of the subject's weight, such as 1 mg/kg and about 2000 mg/kg of the subject's weight, about 100 mg/kg and about 1500 mg/kg of the subject's weight, about 100 μg/kg and about 2000 mg/kg of the subject's weight, about 200 mg/kg and about 1000 mg/kg of the subject's weight, about 200 mg/kg and about 750 mg/kg of the subject's weight, or about 250 mg/kg and about 500 mg/kg of the subject's weight. In some embodiments, subjects are given valproic acid orally at 10 to 60 mg/kg of body weight per day. For example, 10-15 mg/kg of VPA, a VPA analogue, or VPA derivative is administered for two weeks and if well tolerated the dose is increased by 5-10 mg/kg/week to achieve optimal clinical response. In some examples, the daily dose does not exceed 60 mg/kg of body weight and is given for a minimum of 6 months with liver function monitored every two weeks to monthly.

### IV. Clinical Trials

To obtain regulatory approval for the use of VPA, a VPA analogue, or a VPA derivative to treat a muscular disorder, clinical trials are performed. As is known in the art, clinical trials progress through phases of testing, which are identified as Phases I, II, III, and IV.

Initially the VPA, a VPA analogue, or a VPA derivative is evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of the potential therapeutic in the body of the patient. For a Phase I trial, a small group of patients with a muscular disorder are treated with a specific dose of VPA, a VPA analogue, or a VPA derivative. During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to further evaluate the effectiveness and safety of VPA, a VPA analogue, or VPA derivative. In Phase II trials, VPA, a VPA analogue, or a VPA derivative is administered to groups of patients with a muscular disorder using the dosage found to be effective in Phase I trials.

Phase III trials focus on determining how VPA, a VPA analogue, or a VPA derivative compares to the standard, or most widely accepted, treatment. In Phase III trials, patients are randomly assigned to one of two or more “arms”. In a trial with two arms, for example, one arm will receive the standard treatment (control group) and the other arm will receive VPA, a VPA analogue, or a VPA derivative treatment (investigational group).

Phase IV trials are used to further evaluate the long-term safety and effectiveness of VPA, VPA, a VPA analogue, or a VPA derivative. Phase IV trials are less common than Phase I, II and III trials and take place after VPA, VPA, a VPA analogue, or a VPA derivative has been approved for standard use.

### Eligibility of Patients for Clinical Trials

Participant eligibility criteria can range from general (for example, age, sex, type of disease) to specific (for example, type and number of prior treatments, disease characteristics, blood cell counts, organ function). In one embodiment, eligible patients have been diagnosed with a muscular disorder. Eligibility criteria may also vary with trial phase. Patients eligible for clinical trials can also be chosen based on objective measurement of a muscular disorder and failure to respond to other muscular disorder treatments. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I trials usually include 15 to 30 participants for whom other treatment options have not been effective. Phase II trials typically include up to 100 participants who have already received drug therapy, but for whom the treatment has not been effective.

Participation in Phase II trials is often restricted based on the previous treatment received. Phase III trials usually include hundreds to thousands of participants. This large number of participants is necessary in order to determine whether there are true differences between the effectiveness of VPA, a VPA analogue, or a VPA derivative and the standard treatment. Phase III can include patients ranging from those newly diagnosed with a muscular disorder to those with re-occurring signs and/or symptoms associated with a muscular disorder or a muscular disorder that did not respond to prior treatment.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

### Assessment of Patients Prior to Treatment

Prior to commencement of the study, several measures known in the art can be used to first classify the patients. Patients can first be assessed, for example by determining serum CK levels or other indicators of a muscle disorder, such as increased levels of muscle inflammation, apoptosis, muscle loss, myotube hypertrophy, and/or decreased myofibers stability and cell survival.

### Administration of VPA, a VPA Analogue, or a VPA Derivative in Clinical Trials

VPA, a VPA analogue, or a VPA derivative or composition containing such is typically administered to the trial participants orally. A range of doses of the VPA, a VPA analogue, or a VPA derivative can be tested. Provided with information from preclinical testing, a skilled practitioner can readily determine appropriate dosages of VPA, a VPA analogue, or a VPA derivative for use in clinical trials. In one embodiment, a dose range is from about 100 μg/kg and about 5000 mg/kg of the subject's weight, such as 1 mg/kg and about 2000 mg/kg of the subject's weight, about 100 mg/kg and about 1500 mg/kg of the subject's weight, about 100 μg/kg and about 2000 mg/kg of the subject's weight, about 200 mg/kg and about 1000 mg/kg of the subject's weight, about 200 mg/kg and about 750 mg/kg of the subject's weight, about 250 mg/kg and about 500 mg/kg of the subject's weightout 100 μm and about 500 mM. In some embodiments, subjects are given valproic acid orally at 10 to 60 mg/kg of body weight per day. For example, 10-15 mg/kg of VPA, a VPA analogue, or VPA derivative is administered for two weeks and if well tolerated the dose is increased by 5-10 mg/kg/week to achieve optimal clinical response. In some examples, the daily dose does not exceed 60 mg/kg of body weight and is given for a minimum of 6 months with liver function monitored every two weeks to monthly.

### Pharmacokinetic Monitoring

To fulfill Phase I criteria, distribution of the VPA, a VPA analogue, or VPA derivative is monitored, for example, by chemical analysis of samples, such as blood, collected at regular intervals. For example, samples can be taken at regular intervals up until about 72 hours after the start of treatment.

If analysis is not conducted immediately, the samples can be placed on dry ice after collection and subsequently transported to a freezer to be stored at −70° C. until analysis can be conducted. Samples can be prepared for analysis using standard techniques known in the art and the amount of VPA, a VPA analogue, or VPA derivative present can be determined, for example, by high-performance liquid chromatography (HPLC). Pharmacokinetic data can be generated and analyzed in collaboration with an expert clinical pharmacologist and used to determine, for example, clearance, half-life and maximum plasma concentration.

### Monitoring of Patient Outcome

The endpoint of a clinical trial is a measurable outcome that indicates the effectiveness of a compound under evaluation. The endpoint is established prior to the commencement of the trial and will vary depending on the type and phase of the clinical trial. Examples of endpoints include, for example, decline in serum CK levels, inflammation, apoptosis, and muscle loss. For example, at least a 10% reduction in serum CK levels indicates the patient is responsive to the treatment.

The following examples are provided to illustrate certain particular features and/or embodiments. These examples should not be construed to limit the invention to the particular features or embodiments described.

### EXAMPLES

### Example 1

### VPA Activates Akt both In Vitro and In Vivo

This example provides The disclosed results demonstrate for the first time that VPA is an activator of Akt in muscle, both in vitro and in vivo.

### I. Materials and Methods

i. Antibodies and Other Reagents

Valproic acid was purchased from Sigma-Aldrich (St. Louis, Mo.). Rabbit polyclonal antibodies against activated and total Akt, mTOR, ERK, and p70S6k were purchased from Cell Signaling Technology (Danvers, Mass.). Rabbit polyclonal antibodies against the α7A and α7B integrin chain alternative cytoplasmic domains are described in Song, et al. J. Cell Sci. 106:1139-52 (1993). Expression of α7 integrin cytoplasmic domains during skeletal muscle development: alternate forms, conformational change, and homologies with serine/threonine kinases and tyrosine phosphatases, J. Cell Sci. 106:1139-52 (1993). F-20 mouse monoclonal antibody was used to detect myosin heavy chain. Wortmannin was purchased from Cell Signaling Technology. Antibody against collagen Type VI α chain was purchased from Santa Cruz Biotechnology (Santa Cruz, Calif.). Antibody against CD8 was a FITC-labeled rat anti-mouse CD8a antibody (BD Pharmingen, San Diego, Calif.). Horseradish peroxidase (HRP) and FITC-conjugated secondary antibodies were from Jackson ImmunoResearch (West Grove, Pa.). Cell culture reagents and fluorescein di-β-D-galactopyranoside (FDG) were purchased from GIBCO-BRL (Invitrogen, Carlsbad, Calif.). Apoptotic nuclei were stained using the DeadEnd fluorometric TUNEL assay kit (Promega, Madison, Wis.).

ii. Isolation of α7^{+/-} Mouse Myogenic Cells

The α7^{+/-} mice were derived as reported in Flintoff-Dye, et al. Role for the alpha7beta1 integrin in vascular development and integrity, Dev. Dyn. 234:11-21 (2005). These mice were engineered to contain the LacZ gene driven by the α7 integrin promoter. Skeletal muscles from the hind limbs (including gastrocnemius-soleus, quadriceps and tibialis anterior) of a 3-week old α7^{+/-} mouse were removed under sterile conditions, finely minced in Hank's balanced salt solution (HBSS) and incubated in 5 ml of enzyme solution I consisting of 1.5 U/ml collagenase D (0.15 U/mg powder, from Roche Diagnostics, Indianapolis, Ind.), 2.4 U/ml dispase II (neutral protease, grade II, 0.8 U/mg powder, from Roche Diagnostics) and 2.5 mM calcium chloride for 30 minutes at 37° C. with trituration every 5 minutes. The supernatant was decanted into 5 ml of 20% fetal bovine serum (FBS) (Biomeda Corporation, Foster City, Calif.) in HBSS. Enzyme solution II consisting of 0.6 mg/ml trypsin (stock solution 0.5%, from Invitrogen) in HBSS was added to the remaining muscle fragments, incubated for 30 minutes as above, and the cell suspensions were pooled. After passing the cells through a 40 μM nylon mesh, the suspension of cells was centrifuged at 350×g for 10 minutes and the pellet was suspended in 10 ml of growth medium (see below). Pre-plating on non-coated tissue-culture grade plastic plates (Corning, Lowell, Mass.) for 30 minutes was done to separate myogenic and non-myogenic cells. This procedure was repeated three times. Cells were then plated on 1% gelatin-coated plates.

iii. Cell Culture and Treatment

α7^{+/-} myogenic cells were cultured on 1% gelatin-coated plastic plates in Ham's F-10 growth medium (20% FBS, 0.5% chick embryo extract, 5 ng/ml bFGF, 2 mM glutamine, 100 U/ml penicillin and 100 μg/ml streptomycin). After six passages, cells were grown in DMEM containing 20% FBS, 0.5% chick embryo extract, 2 mM glutamine, 100 U/ml penicillin and 100 μg/ml streptomycin. Differentiation was induced by switching the medium to DMEM containing 2% horse serum and antibiotics when cells attained 90% confluence. After 48 hours of differentiation, 2 mM VPA was added and supplied fresh every 24 hours. Wortmannin was used at 100 nM and added fresh every 24 hours.

iv. FDG Based β-galactosidase Fluorescent Assay

α7^{+/-} muscle cells in 96-well plates were induced to differentiate for 48 hours and various concentrations of VPA were then added to six replicate wells for each concentration. Fresh VPA and differentiation medium were added every 24 hours. Control wells received only differentiation medium. After 48 hours of treatment, medium was aspirated and cells were lysed by adding 50 μl water and freeze-thawing. 50 μl of 2× reaction buffer (2μM FDG, 2 mM MgCl₂, 20 mM NaH₂PO4, pH 7.0, 0.2% β-mercaptoethanol) were added per well and incubated for 20 minutes, followed by addition of 2× stop solution (40 mM Tris-acetate, 2 mM EDTA, pH 8.0). The fluorescence in each well was quantified using an Analyst plate reader (Molecular Devices, Sunnyvale, Calif.).

v. Quantification of Myotube Hypertrophy

After 72 hours of exposure to 2 mM VPA, α7^{+/-} myotubes on Lab-Tek chamber slides (Nalge-Nunc International, Rochester, N.Y.) were fixed in ice-cold methanol for 5 minutes, washed thrice in PBS for 5 minutes and blocked with 5% BSA in PBS for 30 minutes. The cells were stained with anti-myosin heavy chain antibody (MF-20, 1:3 in 1% BSA in PBS) and FITC-conjugated donkey anti-mouse secondary antibody (1:200 in 1% BSA in PBS) for 1 hour each, and mounted in Vectashield with DAPI (Vector Laboratories, Burlingame, Calif.). Micrographs of ten fields were taken for VPA-treated and control myotubes using a Leica DMRXA2 microscope (Leica Microsystems, Germany), AxioCam digital camera (Zeiss Inc., Thorwood, N.Y.) and Openlab software (Improvision, Lexington, Mass.). Areas of myosin heavy chain positive myotubes were measured using Openlab software and the numbers of nuclei in each myotube were counted.

vi. Quantification of Apoptosis in Myotubes

Differentiating cultures of α7^{+/-} cells on 1% gelatin-coated Lab-Tek 8-well chamber slides (Nalge-Nunc International) were exposed to 2 mM VPA on day two of differentiation, and fresh VPA in differentiation medium was added every 24 hours. On day eight of differentiation, the DeadEnd Fluorometric TUNEL assay (Promega) was used to label apoptotic nuclei, following the manufacturer's instructions. The numbers of clusters of apoptotic nuclei in each chamber were counted.

vii. Testing Dependence of Akt Activation on PI3K in Myotubes

To test whether Akt activation by VPA in α7^{+/-} myotubes is dependent on PI3K, α7^{+/-} cells were differentiated for 48 hours and treated for 72 hours with 2 mM VPA with or without 100 nM Wortmannin. Fresh drugs were added every 24 hours. Phase-contrast micrographs of the cultures were captured at the end of 72 hours of treatment. To determine status of Akt activation in the cultures, α7^{+/-} myotubes were treated as above and protein extracts were made at the end of 72 hours of treatment using a 2% Triton X-100 extraction buffer (see below). Immunoblotting for phospho- and total Akt were done as detailed below.

viii. Administration of VPA to mdx/utrn^{-/-} Mice

mdx/utrn−/− mice lack both dystrophin and utrophin, and were bred and genotyped as reported in Burkin, et al., Enhanced expression of the alpha 7 beta 1 integrin reduces muscular dystrophy and restores viability in dystrophic mice, J. Cell Biol. 152:1207-18 (2001). 240 mg/kg body weight of VPA was injected intraperitoneally twice daily into mdx/utrn^{-/-} mice (n=9) starting at 3 weeks of age, for a period of 5 weeks. Control mice were injected with saline (n=9). Body-weights were determined daily.

ix. Contracture Assay

On the 35^{th} day of treatment, mdx/utrn^{-/-} mice injected with saline (n=9) or VPA (n=9) were tested for contractures in their hind limbs as follows: mice were placed on a flat table and were lifted by their tails so that their fore-limbs touched the table-surface and their hindquarters were in air. The positions of the hind-limbs were photographed. Normally, mice in this position extend their hind-limbs. The presence of contractures in dystrophic mice prevents full extension. The number of mice in the VPA treated and control groups that failed to extend their hind-limbs were noted.

x. Western Blot Analysis

Cell lysates of differentiating cells on 60 mm dishes were prepared as follows: the cells were washed twice with PBS and 200 μl Triton X-100 lysis buffer (2% Triton X-100, 20 mM Tris-Cl, pH 7.4, 150 mM NaCl, 1 mM EDTA, 1 mM EGTA, 2.5 mM NaPPi, 1 mM β-glycerophosphate, 1 mM sodium vanadate, 1:200 Protease Inhibitor Cocktail (EMD Chemicals, San Diego, Calif.) and 1 mM PMSF) were added. The cells were scraped and transferred into Eppendorf tubes, the cell suspensions were triturated and centrifuged at 8000×g at 4° C. for 10 minutes, and the supernatants were collected. Lysates from mouse skeletal muscle were prepared as follows: the quadriceps muscles were dissected out, snap-frozen in liquid nitrogen and pulverized using a mortar and pestle. The powdered muscles were collected in Eppendorf tubes, 500 μl of Triton X-100 lysis buffer were added, rotated at 4° C. for 30 minutes, centrifuged at 8000×g for 10 minutes and the supernatants were collected. Protein concentrations were determined by the Bradford assay. SDS extracts of muscle were made by boiling powdered muscle in extraction buffer (100 mM Tris-Cl, pH 8.0, 10% SDS, 10 mM EDTA and 10% glycerol) for 10 minutes, vortexing vigorously, and centrifuging at 8000×g for 10 minutes. The supernatants were collected and protein concentrations were determined spectrophotometrically at OD₂₆₀ and OD₂₈₀. SDS-PAGE was carried out using 50 μg of protein for each sample and separated proteins were transferred to nitrocellulose membranes. For western blotting using antibodies against signaling proteins, membranes were blocked in 5% BSA in TBST for 2 hours at room temperature. For all other blots, incubations were done in 5% milk in TBST overnight at 4° C. The membranes were incubated overnight at 4° C. with primary antibodies diluted 1:1000 in 1% BSA in TBST. After washing in 1% BSA in TBST, blots were incubated for 1 hour in HRP-conjugated secondary antibodies diluted 1:5000 in 1% BSA in TBST. Blots were processed using the ECL kit (Amersham Biosciences, Piscataway, N.J.) and bands were visualized with Kodak Biomax MR film (Kodak, Rochester, N.Y.). ImageQuant analysis software (Molecular Dynamics, Sunnyvale, Calif.) was used to quantify band intensity.

xi. Quantification of Evan's Blue Dye Uptake

On the 35^{th} day of injection of VPA, mice were injected intraperitoneally with 100 mg/kg body weight of Evan's blue dye. After 24 hours, the quadriceps muscles were collected, frozen in liquid nitrogen-cooled isopentane and stored at −80° C. 8 μm cryosections of quadriceps were fixed in ice-cold methanol for 1 minute and washed in PBS three times for 5 minutes each. To delineate muscle fibers, the sections were incubated for 10 minutes at room temperature in FITC-conjugated wheat-germ agglutinin (WGA) (Invitrogen) diluted 1:500 in PBS. The sections were washed three times in PBS, 5 minutes each time, and the percentage of Evan's blue dye positive fibers in three sections taken 40 μm apart were counted (approximately 5000 fibers total) for each mouse.

xii. Quantification of CD8 positive Cells in Muscle

8 μm thick cryosections of the quadriceps muscles of saline and VPA injected mice were made. Cytotoxic T-cells were detected with a FITC-labeled rat anti-mouse CD8a antibody (BD Pharmingen, San Diego, Calif.) as follows: the sections were fixed in 4% paraformaldehyde for 5 minutes, washed in PBS three times for 5 minutes each, blocked with 5% BSA/PBS for 1 hour, anti-CD8 antibody added at 1:1000 dilution for 1 hour and washed in 1% BSA/PBS three times for 5 minutes each. The sections were mounted in Vectashield containing DAPI (Vector Laboratories). The number of CD8 positive cells and the total number of nuclei in the same field were counted in ten random fields for each mouse. The ratio of the number of CD positive cells to the total number of nuclei were calculated, averaged and plotted for the saline and VPA treated groups.

xiii. Masson's Trichrome Staining

Masson's trichrome staining kit (American MasterTech Scientific Inc., Lodi, Calif.) was used to stain 8 μm muscle cryosections, as per the manufacturer's instructions.

### II. Results

i. Muscle Cells Treated with VPA have Enhanced Levels of α7 Integrin

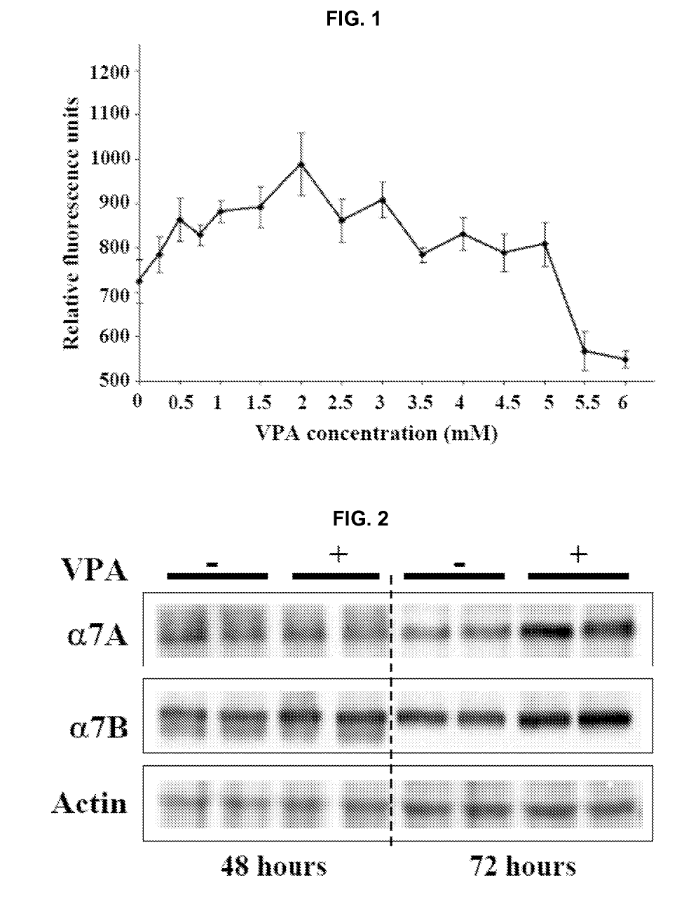

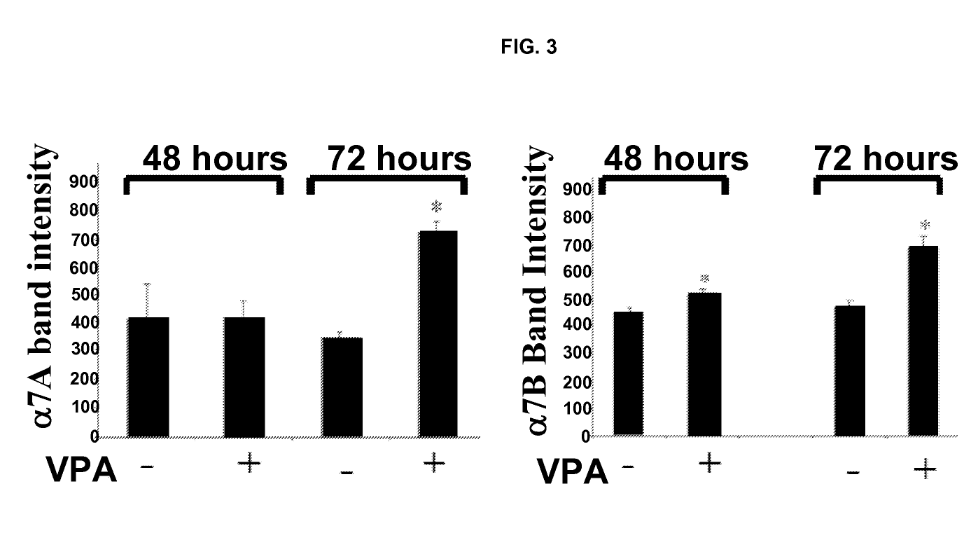

α7^{+/-} myotubes were treated with various concentrations of VPA for 48 hours starting at day two of differentiation. β-galactosidase, the reporter for α7 expression in these cells was quantified using the FDG assay. The effect of VPA, reported by β-galactosidase activity, was concentration-dependent, with the highest level being 1.4-fold, at 2 mM VPA (FIG. 1, data represent the mean±the standard deviation of triplicate samples). Western blotting confirmed increases in α7 protein, FIG. 2. The α7A and α7B integrin isoforms were increased 2-fold and 1.5-fold respectively in myotubes treated with 2 mM VPA for 72 hours (FIG. 3, data represent mean±the standard deviation of duplicate samples. * indicates P<0.05.)

ii. VPA Promotes Hypertrophy and Inhibits Apoptosis in α7^{+/-} Myotubes

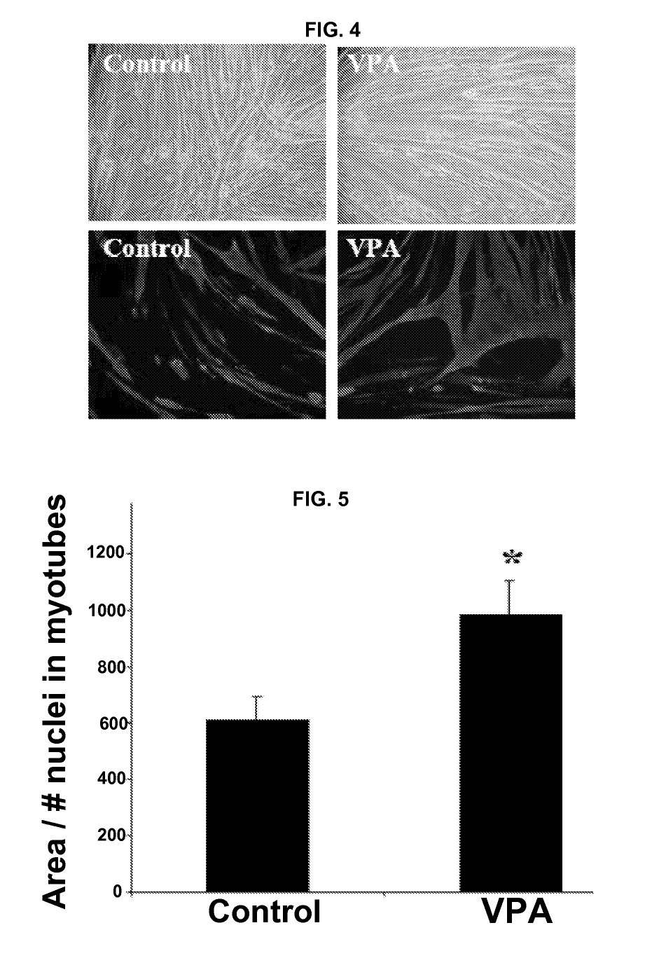

α7^{+/-} myotubes were observed every 24 hours after treatment with 2 mM VPA. As shown in FIG. 4, myotubes in VPA treated cultures were larger compared to controls. To quantify this effect, myotubes were stained with anti-myosin heavy chain antibody, and their areas were determined. To score for hypertrophy, the number of nuclei in the myotubes were counted and the ratios of myotube area to number of nuclei within the myotube were determined. The areas per nucleus of the VPA treated myotubes were 1.6-fold greater than control indicating that VPA promotes hypertrophy in myotubes (FIG. 5, data represent mean±the standard deviation of the ratios in ten random fields. * indicates P<0.05.) The VPA treated cultures also had greater numbers of nuclei per myotube compared to untreated controls, indicating that increased fusion of myoblasts into myotubes also contributed towards forming the larger myotubes.

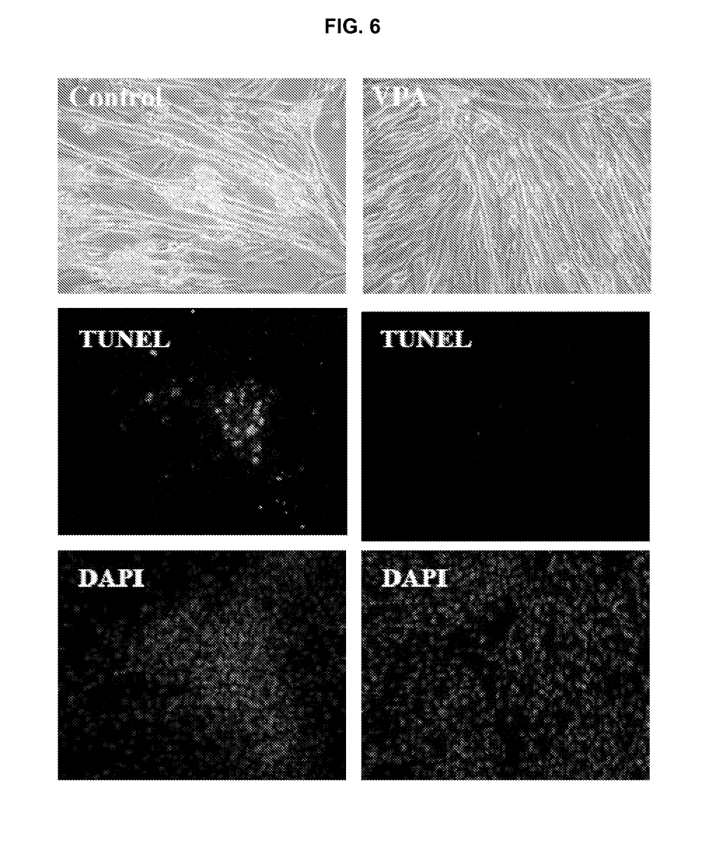

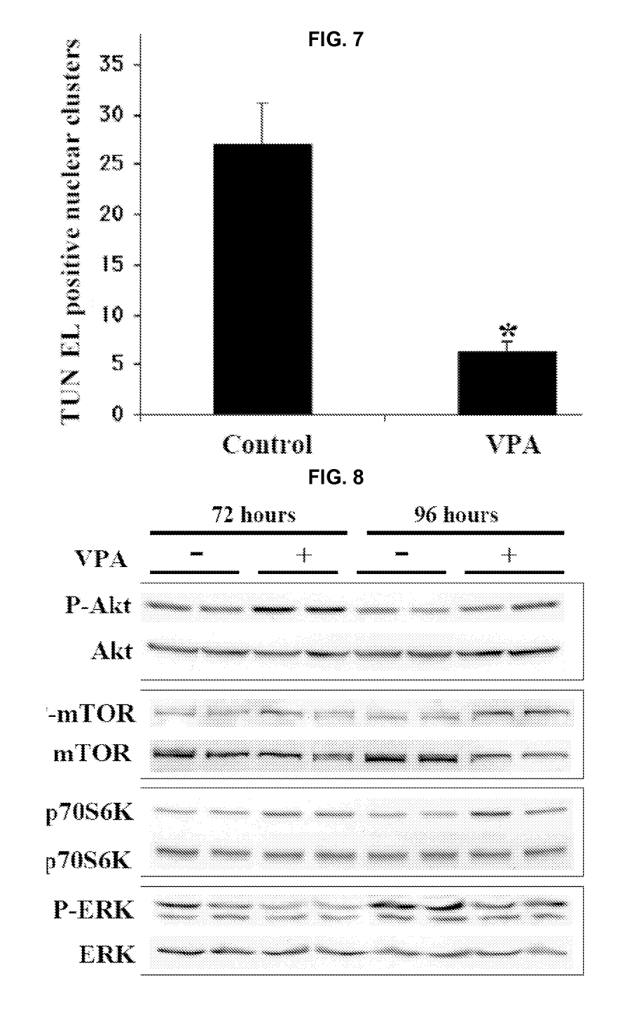

Myotubes kept for prolonged periods of time in culture often become vacuolated, undergo apoptosis and detach. After day 8, α7^{+/-} myotubes in control cultures exhibited vacuoles and some detachment. In contrast, VPA treated cultures appeared relatively normal (FIG. 6, upper panel). A TUNEL assay was used to quantify the proportion of apoptotic nuclei in VPA-treated and control cultures. 2 mM VPA was added on day 2 of differentiation. Fresh medium and VPA were added every 24 hours. On day 6 of treatment, TUNEL assays were performed (FIG. 6, middle panel). The DAPI stating shows all nuclei in the same fields (FIG. 6, lower panel). The total number of TUNEL positive nuclear clusters in four chambers each for VPA and control cultures were counted in FIG. 7 (data represent mean±the standard deviation of quadruplicate values. * indicates P<0.05.) VPA-treated cultures had greater than 5-fold fewer apoptotic nuclei compared with control cells, indicating that treatment with VPA promotes myotube survival.

iii. VPA Activates the Akt/mTOR/p70S6K Pathway and Inhibits the ERK Pathway in α7^{+/-} Myotubes

To determine the mechanism by which VPA promotes hypertrophy and survival, the Akt/mTOR/p70S6K pathway was investigated. Myotubes treated with VPA for 48 or 72 hours had higher levels of activated Akt, mTOR and p70S6K (FIG. 8). In myotubes, the ERK and Akt pathways are antagonistic to each other. As shown in FIG. 8, VPA treated myotubes also had significantly lower levels of activated ERK.

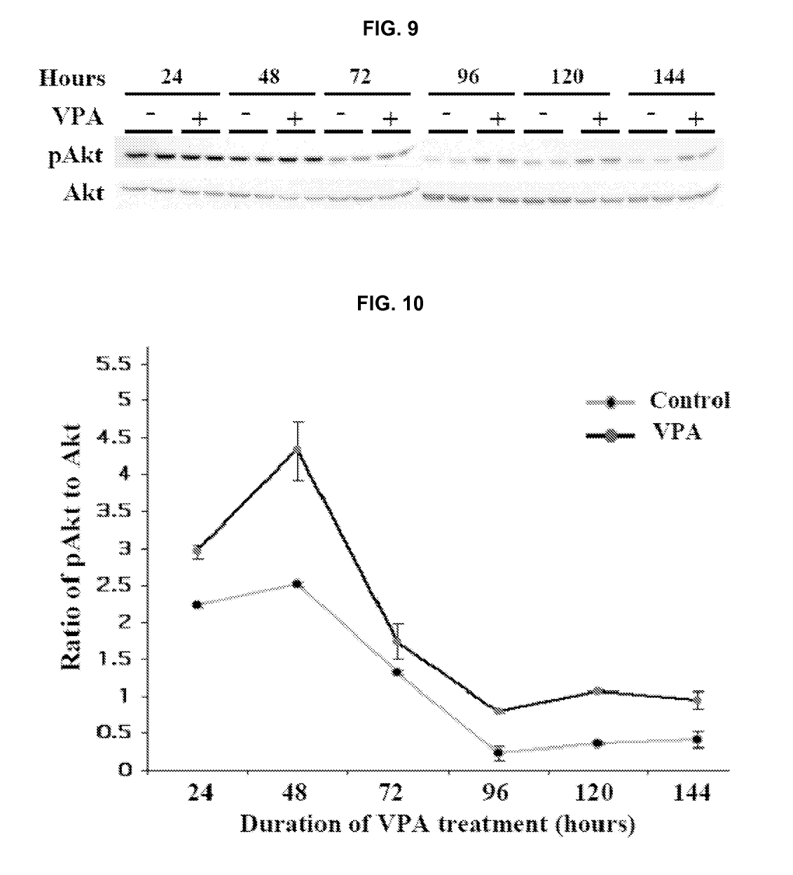

To determine if VPA induced a transient or sustained activation of Akt, 2 mM VPA was added daily to α7^{+/-} myotubes, protein extracts were made every 24 hours up to 144 hours, and western blotting (FIG. 9) was carried out to determine the levels of activated and total Akt. The ratios of band intensities of activated to total Akt were calculated (FIG. 10). VPA treated myotubes exhibited higher levels of activated Akt at all time-points. Although total Akt levels decreased with increasing days of differentiation in both control and VPA treated cultures, the higher proportion of phosphorylated Akt was sustained in the treated α7^{+/-} myotubes.

iv. Akt Activation by VPA is Dependent on PI3K

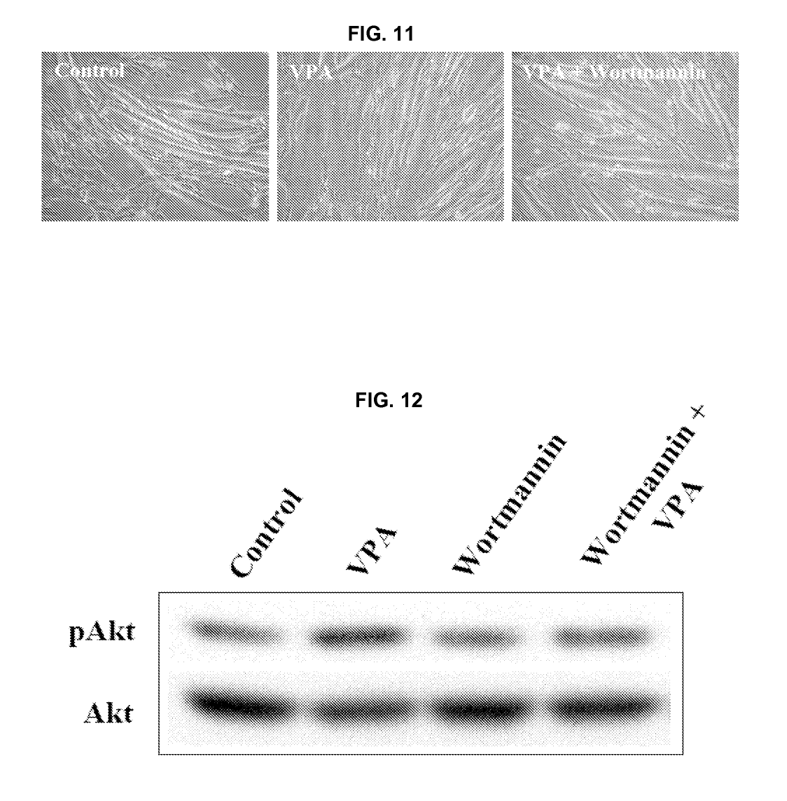

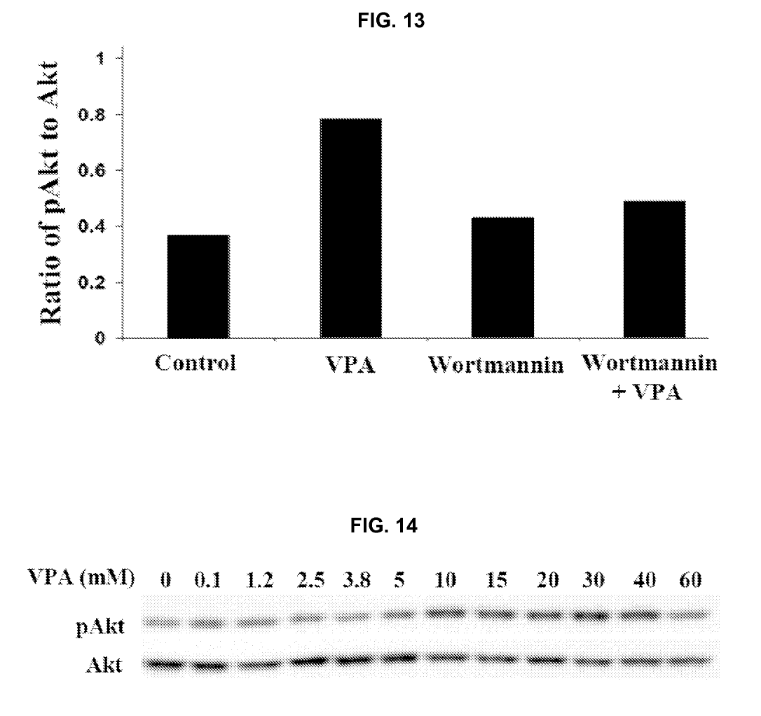

Since VPA has histone deacetylase inhibitor activity, kinases expressed or activated as a result of gene modulation may activate Akt independently of PI3K. Alternatively, VPA may activate Akt through the ‘classical’ PI3K pathway. To differentiate between these possibilities, 2 mM VPA was added on day two of differentiation either with or without 100 nM Wortmannin, a potent inhibitor of PI3K. Fresh VPA and Wortmannin were added every 24 hours thereafter. Microscopic observations revealed that Wortmannin attenuated the myotube hypertrophy promoted by VPA (FIG. 11). Western blots (FIG. 12) of extracts made 72 hours after exposure to the drugs demonstrate that Wortmannin attenuated the activation of Akt by VPA, confirming that VPA activates Akt through PI3K (ratios of phospho to total Akt band intensities plotted in FIG. 13).

v. A One Hour Exposure to VPA Activates Akt and Promotes Myotube Hypertrophy

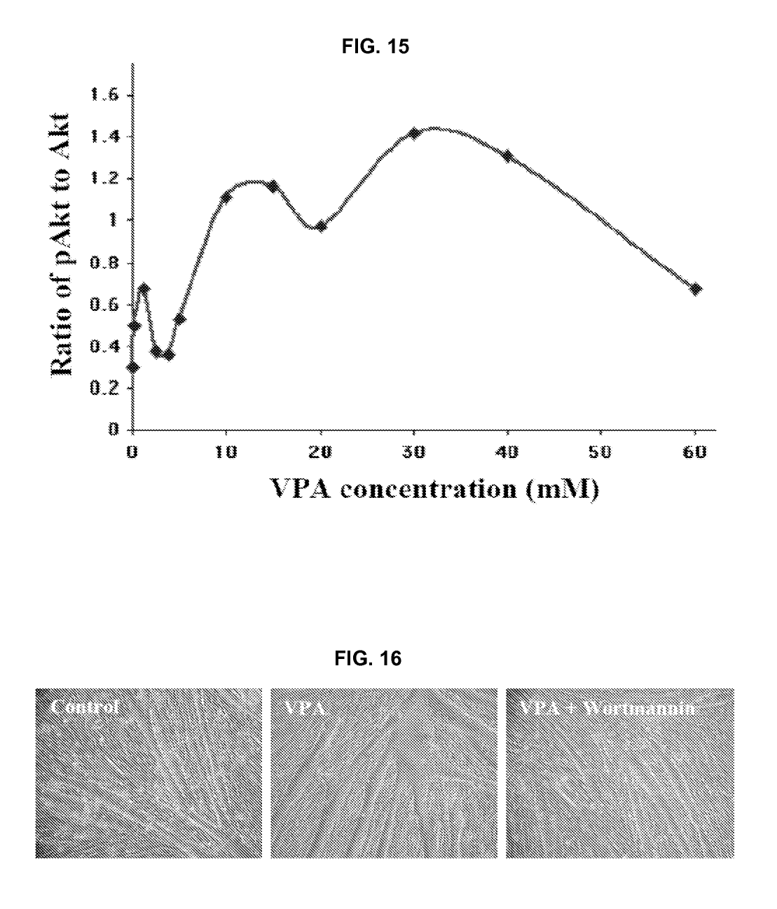

The data above showed that myotubes treated with VPA for 24 hours or longer had higher levels of PI3K mediated Akt activation, leading to hypertrophy and decreased apoptosis. To determine whether this Akt activation and its resulting downstream effects are triggered by VPA at a shorter time after exposure, two-day old differentiating cultures were treated with various concentrations of VPA for 1 hour, extracted and immunoblotted (FIG. 14) for total and phosphorylated Akt. Treatment of α7^{+/-} myotubes with VPA activated Akt within 1 hour. Activation of Akt exhibited a dose-dependent response to VPA, with maximal activation at 30 mM (FIG. 15).

To determine whether Akt activation induced by the 1 hour exposure to VPA is mediated through PI3K and is sufficient to promote myotube hypertrophy, 30 mM VPA, with or without 100 nM Wortmannin was added on day two of differentiation for 1 hour, followed by removal of medium, washing twice with PBS, and adding fresh medium with no VPA or Wortmannin. Microscopic observations 72 hours following this protocol revealed that myotubes exposed to VPA for 1 hour were larger compared to controls (FIG. 16). Thus, a 1 hour exposure of cells to VPA is sufficient to trigger changes that lead to hypertrophy. The presence of Wortmannin inhibited this effect, confirming that the ability of VPA to activate Akt within 1 hour is dependent on PI3K (FIG. 16). These results indicate that Akt activation and the resulting myotube hypertrophy are triggered by VPA at time periods at least as short as 1 hour of treatment.

vi. VPA Ameliorates Hind-limb Contractures in mdx/utrn^{-/-} Mice and Inhibits Fibrosis

As VPA promoted beneficial effects in muscle cells in culture, it was determined if VPA could also have beneficial effects in dystrophic muscle. VPA or saline were administered intraperitoneally into mdx/utrn^{-/-} mice (see Materials and Methods). The mdx/utrn^{-/-} mice exhibit severe muscular dystrophy and reduced lifespan.

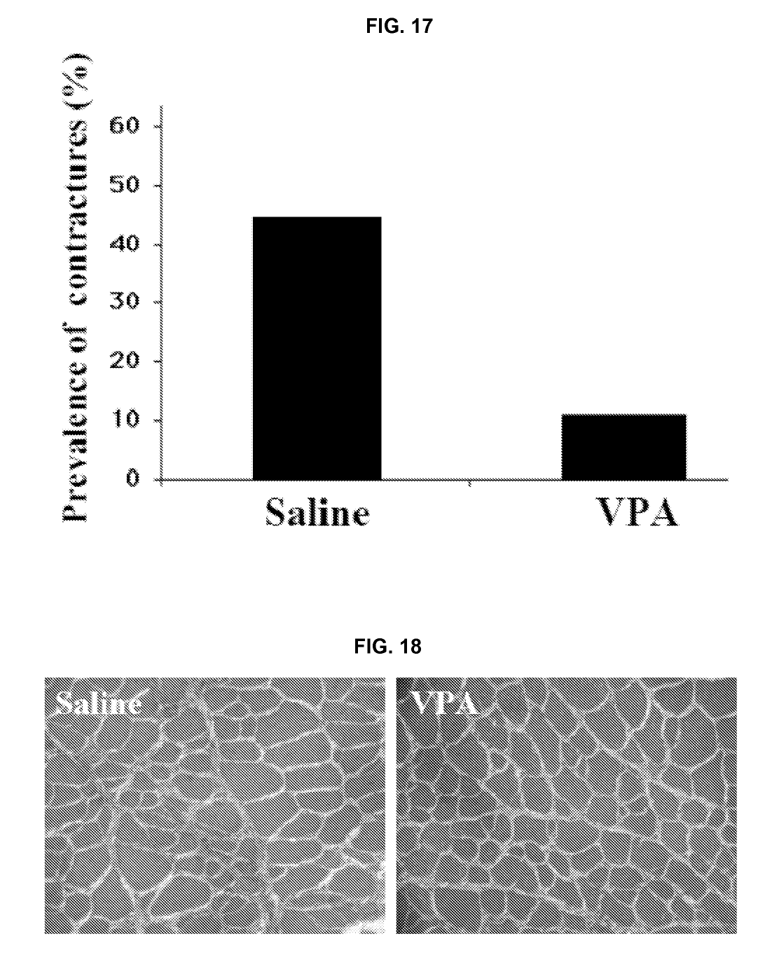

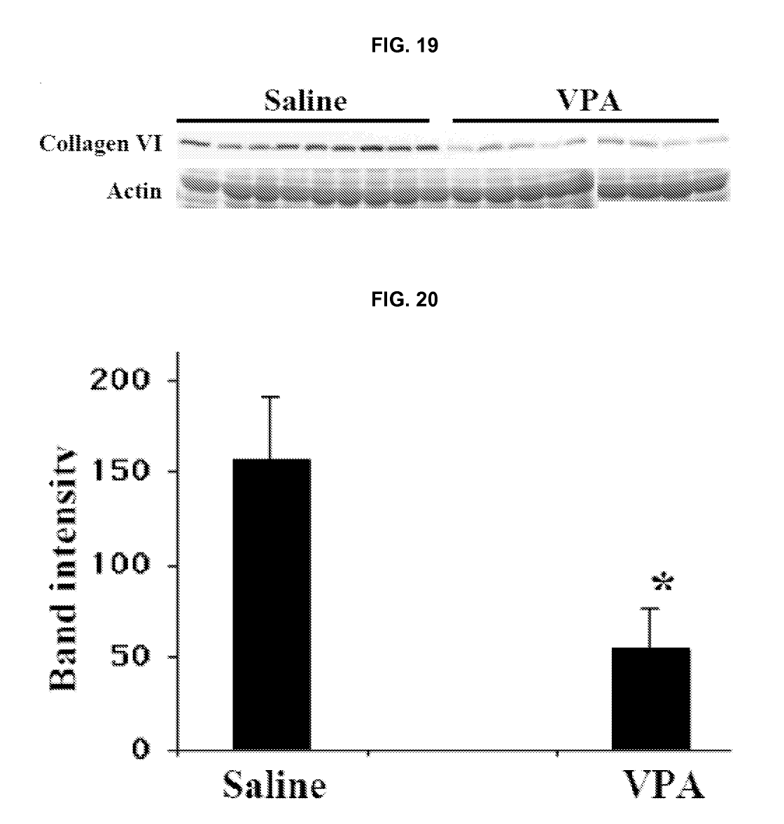

On the 35^{th} day of treatment, the mice were tested for contractures in their hind limbs by lifting of their tails. The presence of contractures prevents full extension of the hind limbs. FIG. 17 illustrates the proportion of mice with contractures in the saline-injected group (4/9, 44.4%) was approximately 4-fold higher compared to the VPA-injected group (1/9, 11.1%). To determine whether treatment with VPA decreased fibrosis, cryosections of quadriceps were stained with Masson's trichrome stain, which stains collagen blue. mdx/utrn−/− mice treated with VPA had a lower intensity of collagen staining compared to controls, indicating that VPA treatment led to decreased collagen content in these muscles (FIG. 18). Type VI collagen is elevated 2 to 3-fold in DMD patients compared to normal. Western blotting (FIG. 19, nine samples each from saline and VPA-injected mice) revealed that mdx/utrn^{-/-} mice treated with VPA had approximately 3-fold less type VI collagen in muscle than control mice (FIG. 20. Data represent mean±the standard deviation. * indicates P<0.05.) Thus, treatment with VPA decreased fibrosis in the mdx/utrn^{-/-} dystrophic mice.

vii. VPA Increases Myofiber Integrity and Decreases Inflammation in Dystrophic Muscle

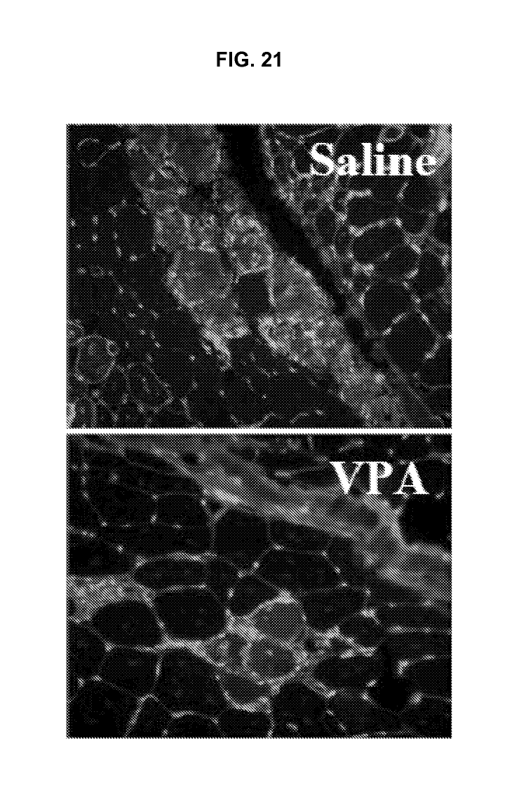

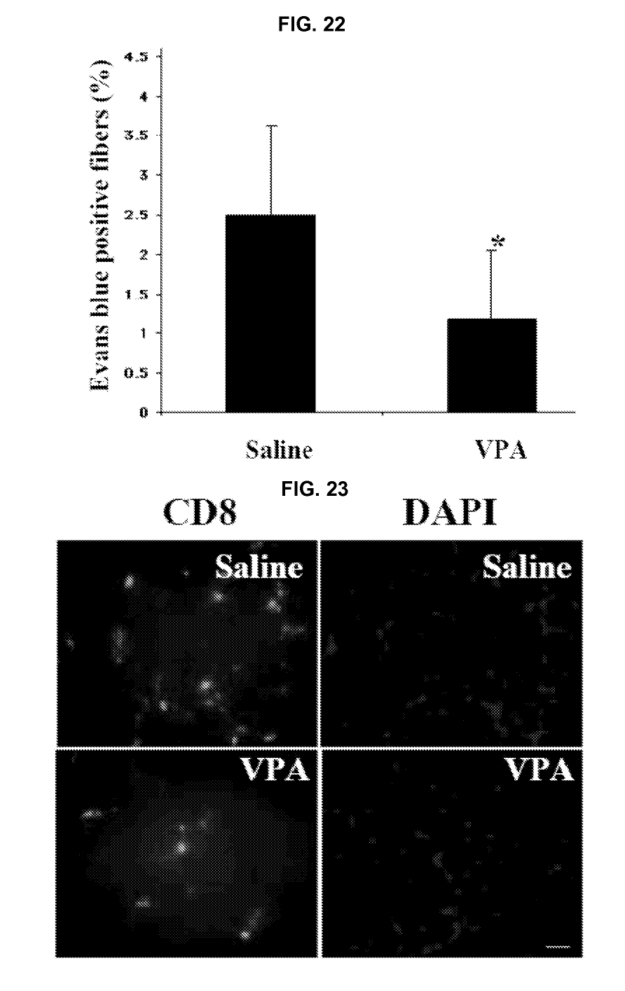

Cryosections from the quadriceps muscles of mice treated with VPA or saline were evaluated for Evans blue dye positive fibers (FIG. 21, nine samples each from saline and VPA-injected mice, red fluorescence shows Evans blue dye positive fibers). The total number of fibers in two sections taken 40 μM apart from each mouse were counted (approximately 5000 fibers for each mouse) and the percentage of Evans blue positive fibers were noted. VPA injected mdx/utrn^{-/-} mice had approximately 2-fold fewer damaged fibers than mice injected with saline (FIG. 22. Data represent mean±the standard deviation. * indicates P<0.05.)

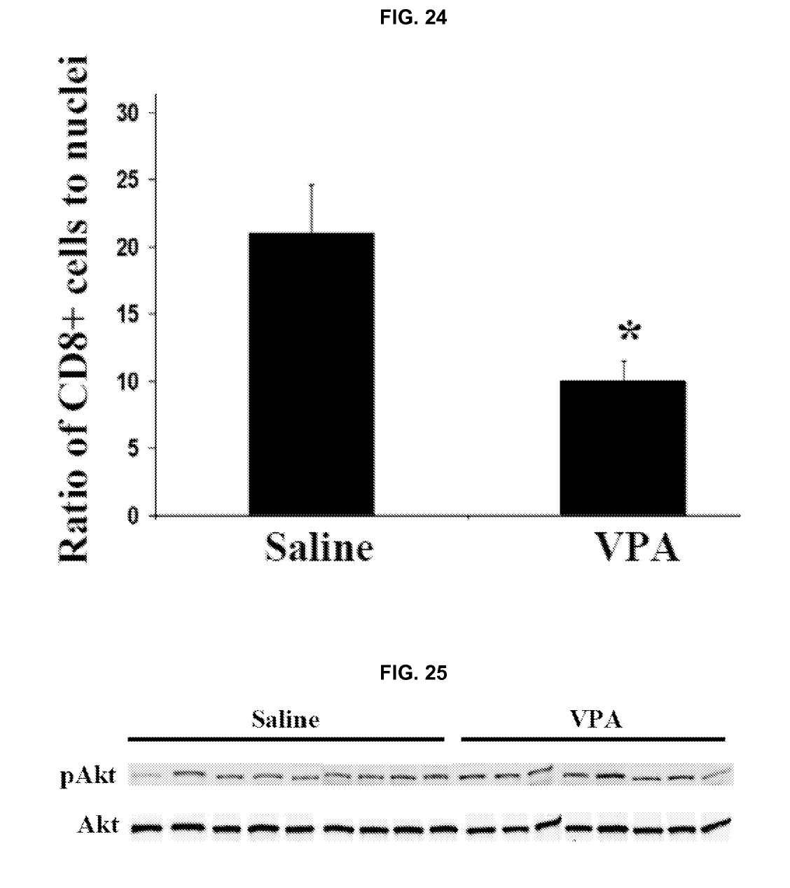

To determine whether VPA reduced inflammation in dystrophic muscle, cryosections from quadriceps of VPA treated and control mdx/utrn^{-/-} mice were stained using anti-CD8 antibody (FIG. 23). The number of CD8 positive cells and the total number of nuclei in the same field were counted in ten random fields for each mouse. The ratio of the number of CD positive cells to the total number of nuclei were calculated, averaged, and plotted for the saline and VPA treated groups (FIG. 24. Data represent mean±the standard deviation. * indicates P<0.05.) The results demonstrate that VPA treatment decreased the CD8 positive cytotoxic T-cell content by approximately 2.1 fold in dystrophic muscle.

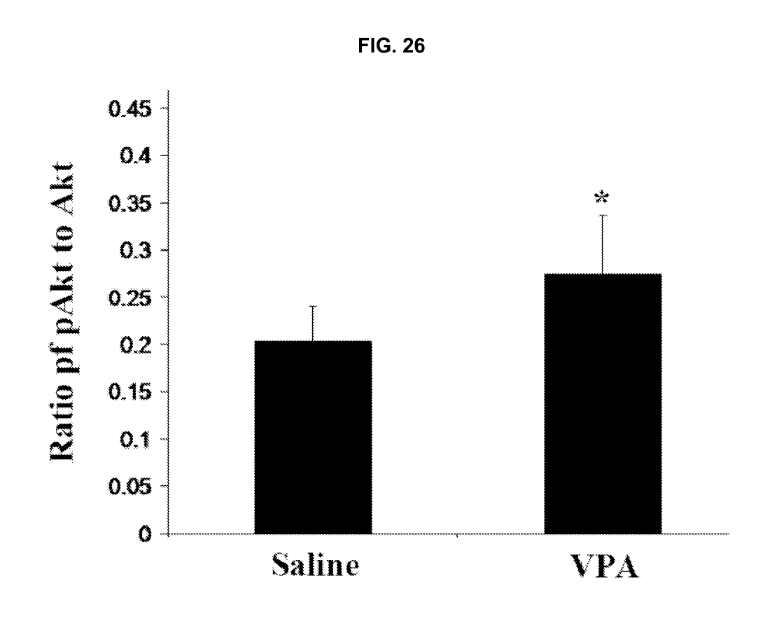

viii. VPA Treated Mice Have Higher Activated Akt in Skeletal Muscle To understand the mechanism by which VPA ameliorated dystrophy in the mdx/utrn^{-/-} mice, western blotting (FIG. 25) was done for activated and total Akt in extracts prepared from quadriceps muscles of saline and VPA treated mice. Significantly higher levels of activated Akt were found in the muscles of VPA treated mice (FIG. 26. Data represent mean±the standard deviation. * indicates P<0.05.) However, western blotting and quantification of α7 integrin in the muscle extracts did not show changes between the saline and VPA injected mice (data not shown).

In summary, these data demonstrate that VPA activates the Akt pathway in myotubes in vitro and in skeletal muscle of severely dystrophic mdx/utrn^{-/-} mice in vivo where it ameliorates dystrophy.

### III. Discussion

Although it is about two decades since mutations in the gene encoding dystrophin were discovered to be the underlying cause of Duchenne muscular dystrophy (DMD), there is still no cure or effective therapy for this devastating neuromuscular disorder. Transgenic over-expression of α7 integrin can partially rescue mdx/utrn^{-/-} mice against dystrophy. Over-expression of α7 integrin in muscle does not disrupt global gene expression profiles.

VPA, a drug that is currently FDA approved for the treatment of epilepsy and bipolar disorders, increased α7 integrin levels in myotubes, maximally at a dose of 2 mM. Microscopic observations revealed that the VPA treated myotubes were larger compared to controls. Myoblasts pre-treated with other HDAC inhibitors have higher rates of fusion into preformed myotubes and thus also produce larger myotubes. However earlier studies show that HDAC inhibitors inhibit myoblast differentiation in vitro. The disclosed results demonstrate that VPA treated cultures had increased ratios of myotube area to number of nuclei, compared to untreated cultures. The number of nuclei per myotube was also increased in VPA treated cultures, indicating that both hypertrophy and increased fusion of myoblasts into myotubes contributed to the larger myotubes seen upon exposure to VPA.

VPA treated myotubes also survived longer in culture compared to untreated controls. Neurons treated with VPA likewise survive for longer periods of time in vitro than untreated controls. To determine whether VPA promoted survival by inhibiting apoptosis, a TUNEL assay was carried out after six days of VPA treatment. VPA treated cultures had 5-fold fewer apoptotic nuclei, indicating VPA promoted myotube survival.

The PI3K/Akt/mTOR pathway was investigated to understand the mechanism by which VPA promotes hypertrophy and inhibits apoptosis. Activation of this pathway promotes hypertrophy and inhibits apoptosis in skeletal muscle. IGF-1 activates this pathway and promotes these effects in muscle and use of IGF-1 has shown encouraging results in treating muscular dystrophy. Immunoblotting with antibodies against activated or total Akt, mTOR and p70S6k showed that VPA activated this pathway in myotubes, thus explaining its hypertrophic and survival effects on myotubes.

To determine if activation of Akt in myotubes is transient or sustained in the presence of VPA, Akt levels were determined every 24 hours starting on day two of differentiation. Higher levels of activated Akt were detected in VPA treated cultures compared to controls at all time points tested, with the proportion of activated Akt increasing with time of exposure to VPA. Beyond 96 hours, there were 2 to 3-fold higher levels of Akt in VPA treated cultures. This may explain the increased survival of myotubes in VPA treated cultures kept for prolonged periods of time.

Wortmannin, a specific inhibitor of PI3K was used to distinguish whether VPA activated Akt through the ‘classical’ PI3K pathway or through an alternate pathway. Wortmannin attenuated Akt activation by VPA, showing Akt activation by VPA is dependent on PI3K. Moreover, Wortmannin inhibited the hypertrophy promoted by VPA, confirming that PI3K mediated Akt activation by VPA is necessary to induce hypertrophy in myotubes.

The disclosed results demonstrate that myotubes treated with VPA for 24 hours or longer had higher levels of PI3K mediated Akt activation, leading to hypertrophy and decreased apoptosis. This activation of Akt and its resulting downstream effects were triggered by treatment with VPA for as short as 1 hour and with a dose-dependent maximal Akt activation at 30 mM. Prior to this disclosure, VPA had not been shown to activate the Akt pathway in muscle. It is suggested herein that VPA activates the PI3K/Akt pathway through direct effects of VPA on PI3K, PDK or both.

Administration of VPA to severely dystrophic mdx/utrn^{-/-} mice led to several beneficial changes in muscle including decreased incidence of hind limb contractures. Masson's trichrome staining also showed decreased collagen in the muscle of VPA treated mice. In DMD patients, type VI collagen is increased approximately 2 to 3-fold above normal and extensive fibrosis contributes to the dystrophic pathology and severely limits the mobility. Western blotting of protein extracted from the quadriceps of mdx/utrn^{-/-} mice treated with VPA showed an approximate 3-fold decrease in levels of this collagen. Mice treated with VPA also had greater myofiber integrity, as shown by decreased Evans blue dye uptake. Furthermore, treatment with VPA decreased the number of CD8 positive inflammatory cells approximately 2.1-fold. These results indicate that VPA promotes increased myofiber integrity that leads to less damage and thus less inflammation, and this eventually results in decreased fibrosis.

To understand the mechanism by which VPA ameliorated dystrophic pathology in mdx/utrn^{-/-} mice, western blotting was done to quantify activated and total Akt in the quadriceps muscles of VPA and saline treated mice. The VPA treated mice had higher activated Akt compared to the saline controls. Studies have shown that Akt activation mediated by IGF-1 plays a central role in inhibiting apoptosis and promoting growth in muscle. Elevated levels of activated Akt are seen in mdx mice that over-express IGF-1 in muscle, and these mice have decreased fibrosis and myonecrosis. The disclosed results demonstrate that VPA-mediated activation of Akt also promotes muscle integrity.

Although transgenic α7mdx/utrn^{-/-} mice that over-express the integrin, and α7^{+/-} myotubes treated in vitro with VPA, both have increased levels of α7 chain and increased activated Akt, there was no change in α7 levels in the muscles of mdx/utrn^{-/-} mice injected with VPA. Thus VPA and α7 integrin appear to activate the Akt pathway differently. VPA mediated activation of Akt appears to be independent of the integrin and the excess integrin present in the α7mdx/utrn^{-/-} mice was sufficient to mediate activation of Akt. In both cases, activation of Akt promoted muscle integrity. The dose and duration of injection of VPA in the mdx/utrn^{-/-} mice may not have been optimal to increase α7 chain levels. The half-life of VPA in mice is about 45 minutes, thus serum levels of VPA may not have been sustained long enough to stimulate α7 integrin expression in the mdx/utrn^{-/-} mice. A different method of VPA administration such as implantable subcutaneus capsules or pumps might be more effective at achieving sustained serum levels of the drug and enhanced α7β₁ integrin levels would likely promote an even greater degree of amelioration of muscle pathology in mice. Additionally, it has recently been shown that α7 integrin promotes activation and proliferation of satellite cells for regeneration in muscle. In humans, the half life of VPA is approximately 14 hours. Thus, therapeutic levels of the drug may be achieved and sustained in DMD patients at lower doses.

The disclosed results demonstrate for the first time that VPA is an activator of Akt in muscle, both in vitro and in vivo. These findings were reported in detail in Gurpur et al., American J. Pathology 174: 999-1008 (2009) which is hereby incorporated by reference in its entirety. As the drug is effective in decreasing fibrosis, increasing myofiber integrity and decreasing inflammation, VPA and its derivatives can be used to treat muscular dystrophy.

### Example 2

### VPA-Mediated Amelioration of Muscle Pathology

This example illustrates VPA-mediated amelioration of muscle pathology in the dyW mouse model of merosin deficient congenital muscular dystrophy.

The congenital muscular dystrophies (CMD) are a diverse group of diseases with clinical features including hypotonia, weakness and contractures with variable disease progression. The incidence of CMD varies between 4.7 and 6.3 per 100,000 live births. Merosin deficient congenital muscular dystrophy (MDC1A) is considered the most common type, accounting for 30-40% of the CMDs.

MDC1A is caused by a mutation in the lama2 gene which encodes the laminin α2 chain. Laminins are heterotrimeric proteins composed of a heavy α chain and two structurally similar light chains (β and γ) and are a major component of the extracellular matrix (ECM). Laminin 111 (α1, β1, γ1) is the predominant isoform found in developing skeletal muscle with laminin 211 (α2, β1, γ1) being the predominant isoform in differentiated skeletal muscle.

In skeletal muscle laminin 211 anchors myofibers to the ECM Laminin α2 deficient myotubes are unstable due to their inability to attach to the ECM and undergo apoptosis. Laminin 211 is also expressed by Schwann cells in the peripheral nervous system, thus lack of laminin 211 leads to deficient myelination, impaired conduction velocity and peripheral neuropathy.

There are several mouse models for MDC1A available. The dy/dy mouse produces a negligible amount of laminin α2 and has a phenotype similar to that of MDC1A, however its use in research has been limited because the disease causing mutation has not been identified. The dyW mouse also has a phenotype similar to MDC1A patients, and the mutation causing laminin α2 deficiency is well-characterized. The dyW mouse has been used in studies investigating pathophysiology of disease and therapeutics. Inhibition of apoptosis leads to improved phenotype in the dyW model. This has been achieved by strategies including inhibition of Bax, overexpression of Bcl-2, or doxycline administration leading to Akt activation.

Small molecules, including VAP, with anti-apoptotic properties can be useful therapeutic agents in MDC1A. VPA activates Akt in neurons and promotes their survival. VPA is also known to have histone deacetylase inhibitor activity. VPA activates the Akt/mTOR/p70S6k pathway in muscle cells in culture and in the mdx/utr(−/−) mouse model of Duchenne muscular dystrophy (Example 1 and Gurpur et al., 2009). Administration of VPA to mdx/utr(−/−) mice conferred multiple beneficial effects in skeletal muscle including increased sarcolemmal integrity, decreased hind-limb contractures and decreased inflammation. Id.

The Akt pathway promotes hypertrophy and survival in skeletal muscle. Akt is activated by the phosphatidyl inositol 3-OH kinase (PI3K), which in turn activates the rapamycin-sensitive kinase mammalian target of rapamycin (mTOR) and downstream targets including p70S6 kinase. This causes gene transcription, protein synthesis and muscle hypertrophy. Activation of the Akt pathway has been explored as a therapeutic option for muscular dystrophy. Cells in culture and mice expressing a constitutively active form of Akt show muscle hypertrophy. The role of Akt in muscle is revealed by the fact that inhibiting mTOR using rapamycin blocks hypertrophy in rodents. Activated Akt also plays a role in inhibiting apoptosis by functioning through multiple signaling proteins including IkB kinase, caspase-9, Bad and forkhead transcription factors.

Based upon the teachings herein, methods of treating CMD (such as MDC1A) are enabled. Also, methods of inhibiting apoptosis, inflammation and increasing cell survival are also enabled. These methods include administration of VPA at a therapeutically-effective concentration to ameliorate one or more of the aforementioned pathologies.

In particular, the effect of VPA on MDC1A is determined by using the dyw mouse model of MDC1A. VPA (60 mg/kg or 240 mg/kg body weight) is administered i.p. daily to male dyw mice (n=11 each, as determined by Power analysis with α=0.05, p=0.8 and r=0.5) for 6 weeks starting at 10 days of age. Daily body weights are noted and mice observed for signs of overt toxicity. Control mice of each genotype will be injected with the same volumes of sterile saline. At the end of the course of injections, mice are sacrificed and the gastrocnemius-soleus, quadriceps, heart and diaphragm harvested. Tissues are sectioned on a Leica CM 1850 cryostat to 10 μm. Hematoxylin-eosin staining is performed on the sections. The effect of VPA on the Akt pathway, fibrosis, apoptosis and/or fiber-diameter variation in skeletal muscle of dyw mice is determined by methods known to those of ordinary skill in the art including, but not limited to those described below.

i. Fiber-diameter Variation

As muscle fiber-diameter variation is a pathological feature in MDC1A muscle, it can be determined if treatment with VPA restores this parameter in dyw mice. Using image analysis software, muscle-fiber diameters are calculated in at least 500 fibers per mouse in both the VPA and control treated groups. If VPA decreases inflammation, levels of CD4 and CD8 will be decreased in mice receiving VPA as compared to that in a control mouse that does not received VPA treatment. Data is analyzed by ANOVA and a p-value of <0.05 is considered statistically significant.

ii. Inflammatory Infiltrate

To quantify inflammatory infiltrate in the muscles, cryosections are processed for immunofluorescence using antibodies against CD4, CD8 and F4/80. The number of cells positive for each of these markers and the total number of nuclei in the same field are counted in 15 random fields for each mouse. The ratio of the number of positive cells to the number of nuclei is calculated, averaged and plotted for the VPA-treated and control mice. If VPA decreases inflammation, levels of CD4 will be decreased in mice receiving VPA as compared to that in a control mouse that does not received VPA treatment. Data is analyzed by ANOVA and a p-value of <0.05 is considered statistically significant.

iii. Fibrosis in Muscle

To quantify fibrosis in muscles, western blotting is performed on muscle extracts using an antibody against collagen VI (Santa Cruz Biotechnology, Santa Cruz, Calif.). Protein extracts are made from muscles as reported (Gurpur et al., 2009) and the Bradford assay is done to determine protein concentrations. Western blotting is carried out using 1:1000 dilution of the anti-collagen VI antibody. The blots are re-probed with an antibody against tubulin to ensure equal protein loading. Band intensities are quantified using the LiCor Odyssey software (LiCor Biosciences, Lincoln, Neb.). Ratio of the collagen VI to tubulin band-intensities is calculated. If VPA decreases fibrosis in muscle, weaker collagen VI band intensities will be measured in samples obtained from mice receiving VPA as compared to that in a control mouse that does not received VPA treatment. Data is analyzed by ANOVA and a p-value of <0.05 is considered statistically significant.

iv. Apoptosis in Muscle

Muscle fiber loss due to apoptosis is a significant problem in MDC1A patients. To determine if VPA treatment decreases apoptosis in dyw muscle, after the course of injections, mice are sacrificed and the gastrocnemius-soleus, quadriceps and diaphragm harvested. Tissues are sectioned on a Leica CM 1850 cryostat to 10 μm. The DeadEnd fluorometric TUNEL assay kit (Promega, Madison, Wis.) is used as per the manufacturer's instructions to label apoptotic nuclei in the cryosections. The total number of nuclei (DAPI labeled, fluorescing blue) and the apoptotic nuclei (fluorescing green) are counted in 15 random fields for each muscle, for each mouse. The proportion of apoptotic nuclei is quantified and compared between the VPA injected and control mice. If VPA decrease apoptosis in muscle, mice receiving VPA will have fewer apoptotic nuclei as compared to that in a control mouse that does not received VPA treatment. Data is analyzed by ANOVA and a p-value of <0.05 is considered statistically significant.

v. Effect of VPA on Prolonging Life-span in dyw Mice

Fifty percent of the dyw mice die prematurely by 4 weeks of age. To determine if VPA treatment prolongs life-span, 20 male dyw mice are injected i.p. daily with 60 mg/kg or 240 mg/kg body weight of VPA starting at 10 days of age. Daily body weights are noted and mice observed for signs of overt toxicity. Control mice are injected with the same volumes of sterile saline. Using Kaplan-Meier analysis, the ability of VPA to improve lifespan is assessed. If VPA prolongs life-span, mice receiving VPA will have a longer lifespan as compared to that in a control mouse that does not received VPA treatment.

vi. Effect of VPA on Activation of the Akt in dyw Skeletal Muscle

VPA is administered to male dyw mice (n=11 each) daily for 6 weeks with 80 mg/kg or 240 mg/kg body weight of VPA starting at 10 days of age. Daily body weights are noted and mice are observed for signs of overt toxicity. Control mice are injected with the same volumes of sterile saline.

After the course of injections, mice are sacrificed and the gastrocnemius-soleus, quadriceps and diaphragm harvested. Protein extracts are made from the muscles as reported (Gurpur et al., 2009). After determining protein concentrations using the Bradford assay, equal protein is loaded on SDS-PAGE gels and western blotting will be carried out using antibodies against the phosphorylated members of the Akt pathway including Akt (both the phospho-Ser473 and -Thr308 antibodies), mTOR and p70S6 kinase. After probing with the phospho-specific antibody, blots are re-probed with antibodies that recognize both the active and inactive forms of the respective proteins. Antibodies are purchased from Cell Signaling Technology (Danvers, Mass.). Alexa Fluor 680-conjugated secondary antibodies are used to probe the primary antibodies, and the blots scanned using an Odyssey infrared imager (Licor Biosciences, Lincoln, Neb.). Band intensities are quantified using the ImageQuant analysis software (Amersham). Data are analyzed by ANOVA and a p-value of <0.05 is considered statistically significant.

vii. Effect of VPA on Improving Muscle Strength in dyw Muscle

To determine if VPA treatment improves muscle function in dyW mice, VPA is administered to male dyW mice (n=11 each) as above. Daily body weights are noted. Control mice of each genotype are injected with the same volumes of sterile saline.

The mice are then assessed for fore-limb grip strength using a SDI Grip Strength System and a Chatillon DFE Digital Force Gauge (San Diego Instruments, Inc., San Diego, Calif.). Mice are allowed to grasp a horizontal platform with their forelimbs and pulled backwards. The peak tension (grams of force) is recorded on a digital force gauge as mice release their grip. Six consecutive tests are performed for each mouse and the data will be averaged. If VPA improves muscle strength in dyW mice, the peak tension will increase as compared to that in a control mouse that does not received VPA treatment. Data are analyzed by ANOVA with a p-value <0.05 considered statistically significant.

viii. Effect of VPA on Improving Behavior in dyw Mice

Normally, mice introduced to a new cage move about in the cage and stand up on their hind limbs. Quantification of the number of times a dyw mouse stands up when kept in a new cage has been used as a measure of therapeutic intervention (Girgenrath et al, 2009). To determine if VPA treatment improves behavior in dyw mice, VPA is administered to male dyw mice (n=11 each) as described above. Daily body weights are noted. Control mice of each genotype are injected with the same volumes of sterile saline.

At the end of the 6 weeks of injections, mice are placed in a fresh cage and the number of times each one stands up on its hind limbs in 5 minutes is noted. If VPA improves behavior in dyw mice, the number of times that a mouse stands up is increased as compared to that in a control mouse that does not received VPA treatment. Data is analyzed by ANOVA and a p-value of <0.05 is considered statistically significant.

Based on the results proved in Example 1, it is predicted that the administration of VPA into the dyw mouse model of MDC1A will ameliorate pathology in these mice. It is predicted that VPA activates the Akt pathway, thereby decreasing fibrosis, inhibiting apoptosis and decreasing fiber-diameter variation in skeletal muscle of dyw mice, thereby ameliorating one or more symptoms associated with MDC1A.

Although this particular example has concentrated on evaluating the effect of VPA in the dyw mouse model of MDC1A, those of skill in the art will appreciate that similar studies can be performed to determine the effect of VPA on other types of muscular dystrophies as well.

### Example 3

### Therapeutic use of Valproic Acid in Cells from MDC1A Patients

This example illustrates the possible therapeutic use of VPA in cells from MDC1A patients.

The effect of VPA on promoting laminin-alpha2 expression in fibroblasts/myoblasts from MDC1A patients is determined by culturing control and MDC1A patient fibroblasts or myoblasts and treatins such with VPA at 0, 0.5 mM, 1 mM, 2 mM and 4 mM for 48 and 72 hours in triplicate. Laminin-alpha2 production from cells was then determined by any of the following assays: (1) immunofluorescence using anti-laminin-alpha2 antibody; (2) ELISA to detect human laminin; (3) RT-PCR to detect laminin-alpha2 chain; and (4) Western analysis to detect laminin-alpha2 chain. Data is analyzed by ANOVA to compare untreated and treated cells and between control and MDC1A cells. A 2-fold increase in the expression of laminin-alpha 2 indicates that VPA is capable of promoting laminin-alpha2 expression and promotes cell survival through activation of the Akt signaling pathway in muscle.

### Example 4

### Clinical Trial for Use of VPA

This example provides a clinical trial for use of VPA as a therapy for MDC1A and DMD.

The objective of this clinical trial is to assess the efficacy of VPA as a therapeutic for MDC1A and DMD. DMD or MDC1A patients along with normal individuals (individuals that do not have muscular dystrophy) are recruited. Individuals are sorted into control or treatment groups in a double blind manner. Before beginning the trial a full medical history (including current prescription and non-prescription medications and alcohol use) and complete blood work, serum biochemistry, liver function panels and serum creatine kinase are assayed for each individual.

Patients are given valproic acid orally at 10 to 60 mg/kg of body weight per day or a placebo tablet in a double blind manner. Treatment group receives an initial dose of 10-15 mg/kg of VPA for two weeks and if well tolerated the dose is increased by 5-10 mg/kg/week to achieve optimal clinical response. If stomach upset develops, the dose may be increased more slowly. The daily dose will not exceed 60 mg/kg of body weight and is given for a minimum of 6 months with liver function monitored every two weeks to monthly. Subjects are assayed for serum creatine kinase and muscle strength weekly. Hepatic function and therapeutic drug level are monitored every two weeks to monthly.

A minimum 10-20% decrease in serum creatine kinase as compared with control subjects indicates reduced muscle damage and a responder to treatment. Further, a minimum 5-10% increase in muscle strength would be considered significant improvement.

It is to be understood that the above discussion provides a detailed description of various embodiments. The above descriptions will enable those skilled in the art to make many departures from the particular examples described above to provide apparatuses constructed in accordance with the present disclosure. The embodiments are illustrative, and not intended to limit the scope of the present disclosure. The scope of the present disclosure is rather to be determined by the scope of the claims as issued and equivalents thereto.

1. A method of activating Akt in a subject, comprising:
administering to a subject a therapeutically effective amount of an active agent, the active agent being valproic acid, a valproic acid derivative, a valproic acid analogue, or a combination thereof, thereby activating Akt in the subject.

2. The method of claim 1, wherein the subject has a condition characterized by impaired production of a component of the muscle membrane-cytoskeleton-extracellular matrix complexes.

3. The method of claim 1, wherein the subject has impaired production of dystrophin.

4. The method of claim 1, wherein the active agent is administered with an additional therapeutic agent, wherein the additional therapeutic agent is laminin, laminin derivative, or laminin analogue, a costameric protein, a growth factor, satellite cells, stem cells, myocytes or a combination thereof.

5. (canceled)

6. The method of claim 1, wherein the active agent is administered in an amount of between about 100 μg/kg and about 5000 mg/kg of the subject's weight.

7. (canceled)

8. The method of claim 1, wherein the active agent is administered in an amount of between about 100 mg/kg and about 1500 mg/kg of the subject's weight.

9. (canceled)

10. The method of claim 1, wherein the active agent is administered in an amount of between about 200 mg/kg and about 1000 mg/kg of the subject's weight.

11. The method of claim 1, wherein the active agent is administered in an amount of between about 200 mg/kg and about 750 mg/kg of the subject's weight.

12. The method of claim 1, wherein the active agent is administered in an amount of between about 250 mg/kg and about 500 mg/kg of the subject's weight.

13. The method of claim 1, wherein the active agent is administered in a concentration of between about 100 μm and about 500 mM.

14. (canceled)

15. The method of claim 1, wherein the active agent is administered in a concentration of between about 5 mM and about 50 mM.

16. The method of claim 6, wherein an effective amount is an Akt-activating amount.

17. The method of claim 6, further comprising administering the effective amount twice daily.

18. The method of claim 6, further comprising administering the active agent such that the effective amount is provided the equivalent of twice daily.

19. The method of claim 6, wherein the active agent is provided in an extended release composition.

20. The method of claim 6, further comprising administering the active agent such that the effective amount is provided the equivalent of once daily.

21. The method of claim 1, further comprising identifying the subject as suffering from a disease, disorder, or condition responsive to Akt activation.

22. The method of claim 1, further comprising identifying the subject as suffering from muscular dystrophy.

23. The method of claim 1, further comprising identifying the subject as suffering from Duchenne muscular dystrophy, congenital muscular dystrophy, Limb-girdle muscular dystrophy, or facioscapulohumeral muscular dystrophy.

24. The method of claim 1, wherein the active agent is not administered to increase SMN production, not administered to act as a deacetylase inhibitor or a combination thereof.

25. (canceled)

26. (canceled)
